(19) United States
(12) Patent Application Publication
(10) Pub. No.: US 2022/0104755 A1
(43) Pub. Date: Apr. 7, 2022

(54) DIGITAL BIOMARKER

(71) Applicant: Hoffmann-La Roche Inc., Little Falls, NJ (US)

(72) Inventors: Christian GOSSENS, Basel (CH); Michael LINDEMANN, Schopfheim (DE); Florian LIPSMEIER, Basel (CH); Detlef WOLF, Grenzach-Wyhlen (DE)

(73) Assignee: Hoffmann-La Roche Inc., Little Falls, NJ (US)

(21) Appl. No.: 17/553,724

(22) Filed: Dec. 16, 2021

Related U.S. Application Data
(63) Continuation of application No. PCT/EP2020/066661, filed on Jun. 17, 2020.

(30) Foreign Application Priority Data
Jun. 19, 2019 (EP) 19181093.6

(51) Int. Cl. A61B 5/00; G16H 40/63

(52) U.S. Cl. CPC A61B 5/4538; A61B 5/0022; A61B 5/4839; G16B 50/20; A61B 5/7275; G16H 40/63; A61B 5/6898

(57) ABSTRACT

Aspects described herein relate to the field of disease tracking and diagnostics. Specifically, they relate to a method of assessing a muscular disability and, in particular, spinal muscular atrophy (SMA) in a subject comprising the steps of determining at least one parameter from a dataset of sensor measurements of the subject using a mobile device, and comparing the determined at least one parameter to a reference, whereby the muscular disability and, in particular, SMA will be assessed. Aspects described herein also relate to a mobile device comprising a processor, at least one pressure sensor and a database as well as software which is tangibly embedded to said device and, when running on said device, carries out the method of the invention as well as the use of such a device for assessing a muscular disability and, in particular, SMA.

### CROSS-REFERENCE TO RELATED APPLICATIONS

This application is a continuation of International Application No. PCT/EP2020/066661, filed Jun. 17, 2020, which claims priority to EP Application No. 19181093.6, filed Jun. 19, 2019, which are incorporated herein by reference in their entireties.

### FIELD

Aspects described herein relates to the field of disease tracking and supporting the diagnostics process, in particular of assessing a muscular disability, in particular, spinal muscular atrophy (SMA) in a subject. Aspects described herein also relate to a mobile device comprising a processor, at least one sensor and a database as well as software which is tangibly embedded to said device and, when running on said device, carries out the method as described herein as well as the use of such a device for assessing a muscular disability and, in particular, SMA. Aspects described herein also relate to a computer-implemented method using machine learning to predict the clinical anchor score of a subject, in particular of a patient suffering from a muscular disability and, in particular, SMA.

### BACKGROUND

Spinal muscular atrophy (SMA), in its broadest sense, describes a collection of inherited and acquired central nervous system (CNS) diseases characterized by progressive motor neuron loss in the spinal cord and brainstem causing muscle weakness and muscle atrophy. SMA can be characterized by a degeneration of the alpha motor neurons from the anterior horn of the spinal cord leading to muscular atrophy and resulting in paralysis. This alpha motor neuron degeneration thus substantially compromises the vital prognosis of patients. In healthy subjects, these neurons transmit messages from the brain to the muscles, leading to the contraction of the latter. In the absence of such a stimulation, the muscles atrophy. Subsequently, in addition to a generalized weakness and atrophy of the muscles, and more particularly of those of the trunk, upper arms and thighs, these disorders can be accompanied by serious respiratory problems.

Infantile SMA is the most severe form of this neurodegenerative disorder. Symptoms include muscle weakness, poor muscle tone, weak cry, limpness or a tendency to flop, difficulty sucking or swallowing, accumulation of secretions in the lungs or throat, feeding difficulties, and increased susceptibility to respiratory tract infections. The legs tend to be weaker than the arms and developmental milestones, such as lifting the head or sitting up, cannot be reached. In general, the earlier the symptoms appear, the shorter the lifespan. As the motor neuron cells deteriorate, symptoms appear shortly afterward. The severe forms of the disease are fatal and all forms have no known cure. The course of SMA is directly related to the rate of motor neuron cell deterioration and the resulting severity of weakness. Infants with a severe form of SMA frequently succumb to respiratory complications due to weakness in the muscles that support breathing. Children with milder forms of SMA live much longer, although they may need extensive medical support, especially those at the more severe end of the spectrum. The clinical spectrum of SMA disorders has been divided into the following five groups:

1) Type 0 SMA (In Utero SMA) is the most severe form of the disease and begins before birth. Usually, the first symptom of Type 0 SMA is reduced movement of the fetus that can first be observed between 30 and 36 weeks of pregnancy. After birth, these newborns have little movement and have difficulties with swallowing and breathing and die shortly after birth.

2) Type I SMA (Infantile SMA or Werdnig-Hoffmann disease) presents symptoms between 0 and 6 months; this form of SMA is very severe. Patients never achieve the ability to sit, and death usually occurs within the first 2 years.

3) Type II SMA (Intermediate SMA) has an age of onset at 7-18 months. Patients achieve the ability to sit unsupported, but never stand or walk unaided. Prognosis in this group is largely dependent on the degree of respiratory involvement.

4) Type III SMA (Juvenile SMA or Kugelberg-Welander disease) is generally diagnosed after 18 months. Type 3 SMA individuals are able to walk independently at some point during their disease course but often become wheelchair-bound during youth or adulthood.

5) Type IV SMA (Adult onset SMA). Weakness usually begins in late adolescence in the tongue, hands, or feet, then progresses to other areas of the body. The course of adult SMA is much slower and has little or no impact on life expectancy.

All the forms of spinal muscular atrophy are accompanied by progressive muscle weakness and atrophy subsequent to the degeneration of the neurons from the anterior horn of the spinal cord. SMA currently constitutes one of the most common causes of infant mortality. It equally affects girls or boys in all regions of the world with a prevalence of between 1/6000 and 1/10 000. Although it is classified as a rare disease, spinal muscular atrophy is the second most common inherited disease with an autosomal recessive pattern.

Nusinersen (Spinraza™, FDA approval 2017), Onasemnogene abeparvovec (Zolgensm®, FDA approval 2019), Risdiplam (CAS 1825352-65-5) and Branaplam (CAS 1562338-42-4) are drugs well known for the treatment of SMA. Low levels of survival motor neuron protein (SMN) play a causative role in the pathogenesis of SMA. Consequently, new therapies are being developed to boost levels of this protein, e.g., by replacing or correcting defective SMN1 genes or by modulating the expression of SMN2. A further route includes neuroprotection and strategies targeted to improving muscle strength and function. As the SMN protein plays a critical role in early infancy (when the neuromuscular junction is developing), the putative window for intervention is very early and brief, particularly in patients with type I SMA. A frequent and mobile measurement of clinically relevant features, leading to an objective, sensitive and precise measurement will ultimately give a more complete picture of the disease status of a patient. This will result in a reduction of the assessment burden of the patient and support diagnosis.

In addition to drug treatment, patients suffering from SMA typically require special medical care, in particular with respect to orthopaedics, mobility support, respiratory care, nutrition, cardiology and mental health. Data from the U.S. Defense Military Healthcare System (2003-2012) were studied by Armstrong et al. in order to determine healthcare costs for patients with spinal muscular atrophy. Median total expenditures for SMA patients over the decade studied were more than USD 83,000 vs. a median of approx. USD 4,500 for matched controls. In a subgroup of patients with early diagnosis, the median cost was approx. USD 170,000. (J Med Econ. 2016 August;19(8):822-6)

Currently, assessing the severity and progression of symptoms in a subject diagnosed with a muscular disability, in particular SMA, involves in-clinic monitoring and testing of the subject from time to time, with weeks or even months between visits to the doctor. The clinical anchor measurements for muscular disabilities (MFM scores), in particular SMA, can be found here: http://www.motor-function-measure.org/user-s-manual.aspx.

Since SMA is a clinically heterogeneous disease of the CNS, diagnostic tools are needed that allow a reliable diagnosis and identification of the present disease status and symptom progression and can, thus, aid in accurate treatment.

US 2014/163426 relates to a test for evaluation of a patient's neurological and cognitive function. Merlini et al. MUSCLE AND NERVE, vol. 26, no. 1, July 2002 is concerned with the reliability of hand-held dynamometry in SMA. PCT/EP2018/086192 describes feature tests to assess SMA.

### SUMMARY

One technical problem underlying aspects described herein can be seen in the provision of means and methods complying with the aforementioned needs. One technical problem is solved by the embodiments characterized in the claims and described herein below.

E1 A method of assessing spinal muscular atrophy (SMA) in a subject comprising the steps of:
a) determining at least one parameter from a dataset of sensor measurements from said subject using a mobile device; and
b) comparing the determined at least one parameter to a reference, whereby SMA is assessed from the result of the comparison.

E2 The method of E1, wherein the said at least one parameter is a parameter indicative for distal motor function, central motor function and axial motor function.

E3 The method of any one of E1-E2, wherein the dataset of sensor measurements of the individual motor function comprises data from the measurement the maximal pressure which can be exerted by a subject with an individual finger or for the capability of exerting pressure with an individual finger over time, the measurement the maximal duration of the tone “aaah”, the maximal amount of touching the screen in a defined time period, in particular within 30 sec, the maximal double touch asynchronity, the variability of acceleration after wind, the number of a thing collected, in particular collected coins and/or the maximal turn speed of the hand.

E4 The method of any one of E1-E3, wherein the dataset of sensor measurements of the individual motor function comprises data from the following feature measurements:
i. mean pressure applied,
ii. pitch variability,
iii. median time to hit the screen,
iv. double touch asynchronity,
v. time to draw a shape,
vi. maximum turning speed of the phone,
vii. variability of acceleration (after wind), and/or
viii. number of collected coins.

E5 The method of any one of E1-E4, wherein the dataset of sensor measurements of the individual motor function comprises data from the following feature test:
i. Ring the bell,
ii. Cheer the monster,
iii. Tap the monster,
iv. Squeeze the tomato,
v. Walk the trails,
vi. Turn the phone,
vii. Walk the rope, and/or
viii. Collect the coins.

E6 The method of any one of E1-E5, wherein the dataset of sensor measurements of the individual motor function comprises data from daily or at least from measurements of every other day, in particular wherein the dataset of sensor measurements of the individual motor function comprises data from sensor measurements obtained in the morning.

E7 The method of any one of E1-E6, wherein said mobile device has been adapted for carrying out on the subject one or more of the sensor measurements referred to in any one of claims 3 to 6.

E8 The method of any one of E1-E7, wherein a determined at least one parameter being essentially identical compared to the reference is indicative for a subject with SMA.

E9 A mobile device comprising a processor, at least one pressure sensor and a database as well as software which is tangibly embedded to said device and, when running on said device, carries out the method of any one of E1-E8.

E10 A system comprising a mobile device comprising at least one pressure sensor and a remote device comprising a processor and a database as well as software which is tangibly embedded to said device and, when running on said device, carries out the method of any one of E1-E8, wherein said mobile device and said remote device are operatively linked to each other.

E11 Use of the mobile device according to E9 or the system of E10 for assessing SMA on a dataset of sensor measurements of the individual subject.

E12 A combination of the method according to any one of E1-E8 with a pharmaceutical agent suitable to treat SMA in a subject, in particular a m7GpppX Diphosphatase (DCPS) Inhibitors, Survival Motor Neuron Protein 1 Modulators, SMN2 Expression Inhibitors, SMN2 Splicing Modulators, SMN2 Expression Enhancers, Survival Motor Neuron Protein 2 Modulators or SMN-AS1 (Long Non-Coding RNA derived from SMN1) Inhibitors, more particular Nusinersen, Onasemnogene abeparvovec, Risdiplam or Branaplam.

E13 A pharmaceutical agent suitable to treat SMA in a subject, in particular a m7GpppX Diphosphatase (DCPS) Inhibitors, Survival Motor Neuron Protein 1 Modulators, SMN2 Expression Inhibitors, SMN2 Splicing Modulators, SMN2 Expression Enhancers, Survival Motor Neuron Protein 2 Modulators or SMN-AS1 (Long Non-Coding RNA derived from SMN1) Inhibitors, more particular Nusinersen, Onasemnogene abeparvovec, Risdiplam or Branaplam wherein the subject being treated monitor the subject's disease with a method according to any one of E1-E8.

E14 A method for the treatment of SMA, wherein the method comprise administering a m7GpppX Diphosphatase (DCPS) Inhibitors, Survival Motor Neuron Protein 1 Modulators, SMN2 Expression Inhibitors, SMN2 Splicing Modulators, SMN2 Expression Enhancers, Survival Motor Neuron Protein 2 Modulators or SMN-AS1 (Long Non-Coding RNA derived from SMN1) Inhibitors, more particular Nusinersen, Onasemnogene abeparvovec, Risdiplam or Branaplam to a subject and wherein the method comprises a method according to any one of E1-E8 to monitor the disease of the subject.

E15 A combination of the method according to E13, whereby a determined at least one parameter being better compared to the reference parameter of said patient before said subject received treatment with the pharmaceutical agent.

E16 A computer-implemented method using machine learning to predict the MFM32 score of a subject suffering from SMA.

E17 A computer-implemented method using machine learning to predict the FVC score of a subject suffering from SMA.

E18 The method as referred to in accordance with the aspects described herein includes a method which essentially consists of the aforementioned steps or a method which can include additional steps.

As used in the following, the terms “have”, “comprise” or “include” or any arbitrary grammatical variations thereof are used in a non-exclusive way. Thus, these terms can both refer to a situation in which, besides the feature introduced by these terms, no further features are present in the entity described in this context and to a situation in which one or more further features are present. As an example, the expressions “A has B”, “A comprises B” and “A includes B” can both refer to a situation in which, besides B, no other element is present in A (that is a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element can be present once or more than once typically will be used only once when introducing the respective feature or element. In the following, in most cases, when referring to the respective feature or element, the expressions “at least one” or “one or more” will not be repeated, non-withstanding the fact that the respective feature or element can be present once or more than once.

Further, as used in the following, the terms “particularly”, “more particularly”, “specifically”, “more specifically”, “typically”, and “more typically” or similar terms are used in conjunction with additional/alternative features, without restricting alternative possibilities. Thus, features introduced by these terms are additional/alternative features and are not intended to restrict the scope of the claims in any way. The invention can, as the skilled person will recognize, be performed by using alternative features. Similarly, features introduced by “in an embodiment of the invention” or similar expressions are intended to be additional/alternative features, without any restriction regarding alternative embodiments of the invention, without any restrictions regarding the scope of the invention and without any restriction regarding the possibility of combining the features introduced in such way with other additional/alternative or non-additional/alternative features of the invention.

The method can be carried out on a mobile device by the subject once the dataset of pressure measurements has been acquired, or on a different device. Thus, the mobile device and the device acquiring the dataset can be physically identical, e.g., the same device, or different, e.g., a remotely located device. Such a mobile device may have a data acquisition unit which typically comprises means for data acquisition, i.e. software and/or hardware which detect or measure either quantitatively or qualitatively physical and/or chemical parameters and transform them into electronic signals transmitted to the evaluation unit in the mobile device used for carrying out the method according to the invention. The data acquisition unit may also or alternatively include hardware and/or software which detect or measure either quantitatively or qualitatively physical and/or chemical parameters and transform them into electronic signals transmitted to a device being remote from the mobile device and used for carrying out the method according to aspects described herein. Typically, data acquisition is performed by at least one sensor. It will be understood that more than one sensor can be used in the mobile device, e.g. at least two, at least three, at least four, at least five, at least six, at least seven, at least eight, at least nine or at least ten or even more different sensors. Typical sensors used for data acquisition include sensors such as a gyroscope, magnetometer, accelerometer, proximity sensors, thermometer, humidity sensors, pedometer, heart rate detectors, fingerprint detectors, touch sensors, voice recorders, light sensors, pressure sensors, location data detectors, cameras, sweat analysis sensors and the like. The evaluation unit typically comprises a processor and a database as well as software which is tangibly embedded to said device and, when running on said device, carries out one or more methods as described herein. Such a mobile device may also comprise a user interface, such as a screen, which allows for providing the result of the analysis carried out by the evaluation unit to a user. When separate devices are used, the mobile device can correspond and/or communicate with the device used for carrying out the analytical methods by any means for data transmission. Such data transmission can be achieved by a permanent or temporary physical connection, such as coaxial, fiber, fiber-optic or twisted-pair, 10 BASE-T cables. Alternatively, it can be achieved by a temporary or permanent wireless connection using, e.g., radio waves, such as Wi-Fi, 3G, 4G, LTE, LTE-advanced, 5G and/or Bluetooth, and the like. Accordingly, for carrying out methods as described herein, the only requirement is the presence of a dataset of input measurements obtained from a subject using a mobile device. The said dataset may be transmitted or stored from the acquiring mobile device on a permanent or temporary memory device which subsequently can be used to transfer the data to a second device for carrying out the analytics. The remote device which carries out the method of the invention in this setup typically comprises a processor and a database as well as software which is tangibly embedded to said device and, when running on said device, carries out the method of the invention. More typically, the said device can also comprise a user interface, such as a screen, which allows for providing the result of the analysis carried out by the evaluation unit to a user.

The term “assessing” as used herein refers to determining or providing an aid for diagnosing whether a subject suffers from a muscular disability and, in particular, SMA, or not. As will be understood by those skilled in the art, such an assessment, although preferred to be, might not be correct for 100% of the investigated subjects. The term, however, requires that a statistically significant portion of subjects can be correctly assessed and, thus, identified as suffering from a muscular disability or SMA. Whether a portion is statistically significant can be determined without further ado by the person skilled in the art using various well known statistic evaluation tools, e.g., determination of confidence intervals, p-value determination,

Student's t-test, Mann-Whitney test, etc.. Details can be found in Dowdy and Wearden, Statistics for Research, John Wiley & Sons, New York 1983. Typically envisaged confidence intervals are at least 50%, at least 60%, at least 70%, at least 80%, at least 90%, at least 95%. The p-values are, typically, 0.2, 0.1, 0.05. Thus, the method of the present invention can aid the identification of a muscular disability or SMA by evaluating a dataset of pressure measurements, for example. The term also encompasses any kind of diagnosing, monitoring or staging of SMA and, in particular, relates to assessing, diagnosing, monitoring and/or staging of any symptom or progression of any symptom associated with a muscular disability and, in particular, SMA. Once a proper diagnosis or assessment is made, appropriate treatments can be administered or prescribed. These include without limitation drugs, gene therapies, strategies targeted to improving muscle strength and function, orthopaedics, mobility support, respiratory care, nutrition, cardiology and mental health interventions.

A “muscular disability” as referred to herein is a condition which is accompanied by a disabled muscle function. Typically, such a muscular disability can be caused by a disease or disorder such as muscular atrophy and, more typically, it can be a neuromuscular disease such as spinal muscular atrophy. The term “spinal muscular atrophy (SMA)” as used herein relates to a neuromuscular disease which is characterized by the loss of motor neuron function, typically, in the spinal cord. As a consequence of the loss of motor neuron function, typically, muscle atrophy occurs resulting in an early death of the affected subjects. The disease is caused by an inherited genetic defect in the SMNI gene. The SMN protein encoded by said gene is required for motor neuron survival. The disease is inherited in an autosomal recessive manner.

The term “subject” as used herein relates to animals and, typically, to mammals. In particular, the subject is a primate and, most typically, a human. The subject in accordance with the present invention shall suffer from or shall be suspected to suffer from a muscular disability and, in particular, SMA, i.e. it can already show some or all of the symptoms associated with the said disease.

The term “at least one” means that one or more parameters can be determined in accordance with the invention, i.e. at least two, at least three, at least four, at least five, at least six, at least seven, at least eight, at least nine or at least ten or even more different parameters. Thus, there is no upper limit for the number of different parameters which can be determined in accordance with the method of the present invention. For example, there can be between one and four different parameters per dataset of sensor measurement determined. The parameter(s) may be selected from the group consisting of: peak pressure, integral pressure, pressure profile over time, and oscillations of pressure.

The term “parameter” as used herein can refer to a parameter which is indicative for the capability of a subject to exert finger pressure. For example, the parameter can be selected from the group consisting of: peak pressure, integral pressure, pressure profile over time, and oscillations of pressure. Depending on the type of activity which is measured, the parameter can be derived from the dataset acquired by the pressure measurement performed on the subject. Particular parameters to be used in accordance with the present invention are listed elsewhere herein in more detail.

The term “dataset of sensor measurements” refers to the entirety of data which has been acquired by the mobile device from a subject during measurements of sensors of the mobile device, in particular the smartphone or any subset of said data useful for deriving the parameter.

The term “individual finger strength” as used herein refers to force levels which can be exerted by a finger. This includes the capability of applying a pressure peak, the capability of applying a certain pressure level over time (integral pressure) and/or the capability of maintaining a pressure over time.

In the following, particular envisaged pressure tests and means for measuring by a mobile device in accordance with the method of the present invention are specified.

In an embodiment, the mobile device is, thus, adapted for performing or acquiring a data from a pressure test (so-called “ring-a-bell test”) configured to measure the maximum pressure which can be exerted by a finger of a subject is measured. Moreover, the test may be configured to measure the duration of maximum pressure application. The dataset acquired from such test allows identification of the peak pressure, the integral pressure as well as the pressure profile over time. The test can require calibration with respect to the maximum force which can be applied by a finger of the subject first. Moreover, there are sensor specific limitations which shall be regarded. In order to measure pressure in a range which is below the sensor intrinsic saturation, the test can be configured to avoid application of maximum pressure.

The aforementioned pressure measurements can be made by a mobile device such as a smart phone by using the Force Touch technology or 3 D touch technology. Force Touch technology uses electrodes for sensing force which are lining the edges of a screen of the mobile device. Said electrodes determine the pressure applied to the screen. Accordingly, a test can display certain tasks on the screen which require pressing said screen with the finger thereby applying force in certain strength or over a certain time. The measured parameters from the electrodes are subsequently relayed to an electromagnetic linear actuator that oscillates back and forth. Said actuator produces data for a dataset of force measurements in accordance with the invention. 3D Touch technology works by using capacitive sensors integrated directly into the screen. When a press is detected, these capacitive sensors measure microscopic changes in the distance between the backlight and the cover glass. These data are then combined with accelerometer data and touch sensors data to complete the data of the dataset of force measurements which can be used for determining at least one parameter by a suitable algorithm running on, e.g. an evaluation unit. Further details on a force touch sensor to be typically included in a mobile device used to generate the dataset of force measurements to be used in the method of the present is described in U.S. Pat. No. 8,633,916. 3 D Touch technology force sensors to be typically included in a mobile device used to generate the dataset of force measurements to be used in the method of the present is described in WO2015/106183. Further suitable force measurement sensors to be used in mobile devices are described in any one of EP 2 368 170, U.S. Pat. No. 9,116,569, EP 2 635 957, U.S. Pat. No. 8,952,987 or US2015/0097791.

In another embodiment, the mobile device is adapted for performing or acquiring a data from a further pressure test configured to measure the ability to sustain a controlled amount of pressure via a finger over a defined period of time. The dataset acquired from such test allow identifying the oscillation of pressure and a pressure profile over time. The test can require calibration with respect to a comfort pressure level, i.e. thresholds for the comfort level of pressure can need to be identified first. Moreover, the test shall be configured such that the measurement is carried out below the sensor intrinsic saturation for pressure measurements. The aforementioned pressure measurements can be made by a mobile device such as a smart phone by using the force touch technology or 3 D touch technology as defined elsewhere herein or analogue technology that allows measurement of force or pressure on a touch screen.

Both tests can be implemented on the mobile device by a computer program code which requests that the subject user performs certain tasks which allow for potential calibration and the actual pressure measurements. Typically, such tasks can be masked within an entertaining exercise or game which requires that the subject performs the tasks in a playfully and, thus, comfortable manner on the device. By using said game setup, the tasks can be, in particular, also be performed by children or subjects having impaired cognitive capabilities. Moreover, the gaming character of the test can also improve the overall motivation of the subjects to perform the tests. Typically envisaged examples for the pressure measurement tests are described in the accompanying Examples below in more detail.

It will be understood that the mobile device to be applied in accordance with the present invention can be adapted to perform one or more of the aforementioned force measurement tests. In particular, it can be adapted to perform both tests.

Depending on the mobile device, pressure measurements measuring peak pressure, the capability of applying a certain pressure level over time (integral pressure) and/or the capability of maintaining a pressure over time (pressure profile) can also be performed during other uses of the mobile device where actions are performed which allow for the said pressure measurements (passive tests) to be recorded without the user focusing on it. Typically, if a smart phone is used as a mobile device, the subject (user) will usually perform a variety of touch controlled tasks which involve finger pressure-driven interactions with the screen. Typically, tapping will occur when telephone numbers are dialed or other standard activities are performed, e.g. internet queries are made or the like. The pressure applied by the fingers during performing such tasks can be analyzed over a certain time for calibration purposes and for providing a reference. Typically, peak pressure measurements can be performed during, e.g., tapping tasks such as dialing or the applied pressure can be integrated over a certain time window to yield an integral pressure. Change in the peak force, the integral pressure or a task specific pressure profile with respect to the reference can subsequently be used in the method according to the invention to be applied for investigating the dataset obtained from said (passive) pressure measurements.

Moreover, tapping and other pressure applying activities may occur during the further tests mentioned below. Pressure measurements can also be performed as passive tests during said further tests.

Moreover, the mobile device may be adapted to perform further tests which may be relevant for muscular disabilities like SMA. Accordingly, further data can be processed in the method of the present invention as well. These further data are typically suitable for further strengthening the assessment of SMA or muscular disability in a subject. Particular envisaged tests which investigate distal motor function (e.g., tapping, drawing and pinching abilities of fingers), axial motor function (e.g., lifting, twisting, tightrope and water pouring abilities of the subject), and/or central motor function (e.g., voice abilities) described in more detail below. In addition, surveys on overall well-being and cognitive capabilities can be regarded as well.

Particular envisaged further tests to be implemented on the mobile device for acquiring data which can be typically included into the dataset to be investigated by the method of the invention are selected from the following tests:

(1) Tests for distal motor functions: Tap the monster, Walk the trail, and Squeeze a tomato.

The mobile device can be further adapted for performing or acquiring a data from a further test for distal motor function (so-called “Tap the monster”) configured to measure dexterity and distal weakness of the fingers. The dataset acquired from such test allow identifying the finger speed, precision of finger movements and finger travel time and distance.

The mobile device can be further adapted for performing or acquiring a data from a further test for distal motor function (so-called “Walk the trail”) configured to measure dexterity and distal weakness of the fingers. The dataset acquired from such test allow identifying the precision of finger movements, pressure profile and speed profile.

The aim of the “Walk the trail” test is to assess fine finger control and stroke sequencing. The test is considered to cover the following aspects of impaired hand motor function: tremor and spasticity and impaired hand-eye coordination. The patients are instructed to hold the mobile device in the untested hand and draw on a touchscreen of the mobile device different pre-written alternating shapes of increasing complexity (linear, rectangular, circular, sinusoidal, and spiral; vide infra) with the second finger of the tested hand “as fast and as accurately as possible” within a maximum time of for instance 30 seconds. To draw a shape successfully the patient's finger has to slide continuously on the touchscreen and connect indicated start and end points passing through all indicated check points and keeping within the boundaries of the writing path as much as possible. The patient has maximum of two attempts to successfully complete each of the 6 shapes. Tests may be alternatingly performed with right and left hand. The user may be instructed on daily alternation. The two linear shapes may each have a specific number “a” of checkpoints to connect, i.e “a-1” segments. The square shape may have a specific number “b” of checkpoints to connect, i.e. “b-1” segments. The circular shape may have a specific number “c” of checkpoints to connect, i.e. “c-1” segments. The eight-shape may have a specific number “d” of checkpoints to connect, i.e “d-1” segments. The spiral shape may have a specific number “e” of checkpoints to connect, “e-1” segments. Completing the 6 shapes then implies to draw successfully a total of “(2a+b+c+d+e−6)” segments. One or more of the shapes may optionally be given greater weight than the others, e.g., drawing of the number “8”.

Typical Draw a Shape test parameters of interest:

Based on shape complexity, the linear and square shapes can be associated with a weighting factor (Wf) of 1, circular and sinusoidal shapes a weighting factor of 2, and the spiral shape a weighting factor of 3. A shape which is successfully completed on the second attempt can be associated with a weighting factor of 0.5. These weighting factors are numerical examples which can be changed in the context of the present invention.

1. Shape completion scores:
i. Number of successfully completed shapes (0 to 6) (ΣSh) per test
ii. Number of shapes successfully completed at first attempt (0 to 6) (ΣSh₁)
iii. Number of shapes successfully completed at second attempt (0 to 6) (ΣSh₂)
iv. Number of failed/uncompleted shapes on all attempts (0 to 12) (ΣF)
v. Shape completion score reflecting the number of successfully completed shapes adjusted with weighting factors for different complexity levels for respective shapes (0 to 10) (Σ[Sh*Wf])
vi. Shape completion score reflecting the number of successfully completed shapes adjusted with weighting factors for different complexity levels for respective shapes and accounting for success at first vs second attempts (0 to 10) (Σ[Sh₁*Wf]+Σ[Sh₂*Wf0.5])
vii. Shape completion scores as defined in #1e, and #1f can account for speed at test completion if being multiplied by 30/t, where t would represent the time in seconds to complete the test.
viii. Overall and first attempt completion rate for each 6 individual shapes based on multiple testing within a certain period of time: (ΣSh₁)/(ΣSh₁+SΣh₂+ΣF) and (ΣSh₁+ΣSh₂)/(ΣSh₁+ΣSh₂+ΣF).

2. Segment completion and celerity scores/measures:

(analysis based on best of two attempts [highest number of completed segments] for each shape, if applicable)
i. Number of successfully completed segments (0 to [2a+b+c+d+e−6]) (ΣSe) per test
ii. Mean celerity ([C], segments/second) of successfully completed segments: C=ΣSe/t, where t would represent the time in seconds to complete the test (max 30 seconds)
iii. Segment completion score reflecting the number of successfully completed segments adjusted with weighting factors for different complexity levels for respective shapes (Σ[Se*Wf])
iv. Speed-adjusted and weighted segment completion score (Σ[Se*Wf]*30/t), where t would represent the time in seconds to complete the test.
v. Shape-specific number of successfully completed segments for linear and square shapes (ΣSe_{LS})
vi. Shape-specific number of successfully completed segments for circular and sinusoidal shapes (ΣSe_{CS})
vii. Shape-specific number of successfully completed segments for spiral shape (ΣSe_{S})
viii. Shape-specific mean linear celerity for successfully completed segments performed in linear and square shape testing: C_{L}=ΣSe_{LS}/t, where t would represent the cumulative epoch time in seconds elapsed from starting to finishing points of the corresponding successfully completed segments within these specific shapes.
ix. Shape-specific mean circular celerity for successfully completed segments performed in circular and sinusoidal shape testing: C_{C}=ΣSe_{CS}/t, where t would represent the cumulative epoch time in seconds elapsed from starting to finishing points of the corresponding successfully completed segments within these specific shapes.
x. Shape-specific mean spiral celerity for successfully completed segments performed in the spiral shape testing: C_{S}=ΣSe_{S}/t, where t would represent the cumulative epoch time in seconds elapsed from starting to finishing points of the corresponding successfully completed segments within this specific shape.

3. Drawing precision scores/measures:

(analysis based on best of two attempts[highest number of completed segments] for each shape, if applicable)
i. Deviation (Dev) calculated as the sum of overall area under the curve (AUC) measures of integrated surface deviations between the drawn trajectory and the target drawing path from starting to ending checkpoints that were reached for each specific shapes divided by the total cumulative length of the corresponding target path within these shapes (from starting to ending checkpoints that were reached).
ii. Linear deviation (Devi) calculated as Dev in #3a but specifically from the linear and square shape testing results.
iii. Circular deviation (Dev_{C}) calculated as Dev in #3a but specifically from the circular and sinusoidal shape testing results.
iv. Spiral deviation (Dev_{S}) calculated as Dev in #3a but specifically from the spiral shape testing results.
v. Shape-specific deviation (Dev₁₋₆) calculated as Dev in #3a but from each of the 6 distinct shape testing results separately, only applicable for those shapes where at least 3 segments were successfully completed within the best attempt.
vi. Continuous variable analysis of any other methods of calculating shape-specific or shape-agnostic overall deviation from the target trajectory.

4.) Pressure profile measurement
(1) Exerted average pressure
(2) Deviation (Dev) calculated as the standard deviation of pressure

The mobile device can be further adapted for performing or acquiring a data from a further test for distal motor function (so-called “Squeeze the tomato”) configured to measure dexterity and distal weakness of the fingers. The dataset acquired from such test allow identifying the precision and speed of finger movements and related pressure profiles. The test can require calibration with respect to the movement precision ability of the subject first.

One aim of the Squeeze the tomato test is to assess fine distal motor manipulation (gripping and grasping) and control by evaluating accuracy of pinch closed finger movement. The test is considered to cover the following aspects of impaired hand motor function: impaired gripping/grasping function, muscle weakness, and impaired hand-eye coordination. The patients are instructed to hold the mobile device in the untested hand and by touching the screen with two fingers from the same hand (thumb+second or thumb+third finger preferred) to squeeze/pinch as many round shapes (i.e., tomatoes) as they can during 30 seconds. Impaired fine motor manipulation will affect the number of shapes pinched. Tests will be alternatingly performed with right and left hand. The user will be instructed on daily alternation.

Typical Squeeze a Shape test parameters of interest:

1. Number of squeezed shapes
a) Total number of tomato shapes squeezed in 30 seconds (ΣSh)
b) Total number of tomatoes squeezed at first attempt (ΣSh₁) in 30 seconds (a first attempt is detected as the first double contact on screen following a successful squeezing if not the very first attempt of the test)

2. Pinching precision measures:
a) Pinching success rate (PSR) defined as ΣSh divided by the total number of pinching (ΣP) attempts (measured as the total number of separately detected double finger contacts on screen) within the total duration of the test.
b) Double touching asynchrony (DTA) measured as the lag time between first and second fingers touch the screen for all double contacts detected.
c) Pinching target precision (PTP) measured as the distance from equidistant point between the starting touch points of the two fingers at double contact to the centre of the tomato shape, for all double contacts detected.
d) Pinching finger movement asymmetry (PFMA) measured as the ratio between respective distances slid by the two fingers (shortest/longest) from the double contact starting points until reaching pinch gap, for all double contacts successfully pinching.
e) Pinching finger velocity (PFV) measured as the speed (mm/sec) of each one and/or both fingers sliding on the screen from time of double contact until reaching pinch gap, for all double contacts successfully pinching.
f) Pinching finger asynchrony (PFA) measured as the ratio between velocities of respective individual fingers sliding on the screen (slowest/fastest) from the time of double contact until reaching pinch gap, for all double contacts successfully pinching.
g) Continuous variable analysis of 2a to 2f over time as well as their analysis by epochs of variable duration (5-15 seconds)
h) Continuous variable analysis of integrated measures of deviation from target drawn trajectory for all tested shapes (in particular the spiral and square)

3.) Pressure profile measurement
a) Exerted average pressure
b) Deviation (Dev) calculated as the standard deviation of pressure

(2) Tests for measuring axial motor function: Turn the phone, Walk the rope and Collect the coins

The mobile device can be further adapted for performing or acquiring a data from a further test for axial and proximal motor function motor function (so-called “Turn the phone”) configured to measure upper extremity mobility (e.g., by twisting the mobile device), weakness and fatigue, proximal hypotonia, joint contractures and tremor. For this test, the patient has to hold the phone in the palm of his/her hand and turn the phone screen up and down repeatedly.

The dataset acquired from such test allow identifying the precision and speed and number of twists (rotations of the wrist). The test can require calibration with respect to the movement precision ability of the subject first.

The mobile device can be further adapted for performing or acquiring a data from a further test for axial motor function (so-called “Walk the rope”) configured to measure proximal hypotonia in the upper extremities. The dataset acquired from such test allow identifying the number, size and velocity of correct movements. The test can require calibration with respect to the counterbalance and imbalance abilities of the subject first.

The mobile device can be further adapted for performing or acquiring data from a further test for axial motor function (so-called “Collect the coins”) configured to measure upper extremity mobility (by moving the mobile device), weakness and fatigue. The dataset acquired from such test allow identifying the extend of the axial rotation movement, the speed and the number of movements over time as well as reaction times as response to the progressing game situation (i.e. the ball needs to be alternated by the user between opposing sites of the screen). The test can require calibration with respect to the movement precision ability of the subject first.

(3) Tests for central motor function: Cheer the monster

The mobile device can be further adapted for performing or acquiring a data from a further test for central motor function (so-called “Cheer the monster”) configured to measure proximal central motoric functions by measuring voicing capabilities.

Typically, the aforementioned tests can be implemented on the mobile device as well by a computer program code which requests that the subject user performs certain tasks which allow for calibration and the force measurements. Typically, such tasks can be masked within a game which requires that the subject performs the tasks in a playfully and, thus, comfortable and relaxed manner on the device. By using said game setup, the tasks can be, in particular, also be performed by children or subjects having impaired cognitive capabilities. Moreover, the gaming character of the test can also improve the overall motivation of the subjects to perform the tests. Typically envisaged examples for the aforementioned tests are described in the accompanying Examples below in more detail.

In yet an embodiment of the method of the invention, the mobile device from which the dataset is obtained is configured in addition to the dataset of pressure measurements to provide at least data from at least one of the tests for distal motor function, axial motor function and/or central motor function and, more typically, for any one of these types of data.

The term “mobile device” as used herein refers to any portable device which comprises at least a pressure sensor and data-recording equipment suitable for obtaining the dataset of pressure measurements, or other sensors such as an accelerometer and gyroscope. This can also require a data processor and storage unit as well as a display for electronically simulating a pressure measurement test on the mobile device. Moreover, from the activity of the subject data shall be recorded and compiled to a dataset which is to be evaluated by the method of the present invention either on the mobile device itself or on a second device. Depending on the specific setup envisaged, it can be necessary that the mobile device comprises data transmission equipment in order to transfer the acquired dataset from the mobile device to further device. Some examples of mobile devices according to the present invention are smartphones, portable multimedia devices or tablet computers. Alternatively, portable sensors with data recording and processing equipment can be used. Further, depending on the kind of activity test to be performed, the mobile device shall be adapted to display instructions for the subject regarding the activity to be carried out for the test. Particular envisaged activities to be carried out by the subject are described elsewhere herein and encompass the distal hypotonia tests as well as other tests described in this specification.

Determining at least one parameter can be achieved either by deriving a desired measured value from the dataset as the parameter directly. Alternatively, the parameter can integrate one or more measured values from the dataset and, thus, can be a derived from the dataset by mathematical operations such as calculations. Typically, the parameter is derived from the dataset by an automated algorithm, e.g., by a computer program which automatically derives the parameter from the dataset of activity measurements when tangibly embedded on a data processing device feed by the said dataset.

The term “reference” as used herein refers to a discriminator which allows assessing the muscular disability and, in particular, SMA in a subject. Such a discriminator can be a value for the parameter which is indicative for subjects suffering from the muscular disability and, in particular, SMA or subjects not suffering from the muscular disability and, in particular, SMA.

Such a value can be derived from one or more parameters of subjects known to suffer from the muscular disability and, in particular, SMA. Typically, the average or median can be used as a discriminator in such a case. If the determined parameter from the subject is identical to the reference or above a threshold derived from the reference, the subject can be identified as suffering from the muscular disability and, in particular, SMA in such a case. If the determined parameter differs from the reference and, in particular, is below the said threshold, the subject shall be identified as not suffering from the muscular disability and, in particular, SMA.

Similarly, a value can be derived from one or more parameters of subjects known not to suffer from the muscular disability and, in particular, SMA. Typically, the average or median can be used as a discriminator in such a case. If the determined parameter from the subject is identical to the reference or below a threshold derived from the reference, the subject can be identified as not suffering from the muscular disability and, in particular, SMA in such a case. If the determined parameter differs from the reference and, in particular, is above the said threshold, the subject shall be identified as suffering from the muscular disability and, in particular, SMA.

As an alternative, the reference can be a previously determined parameter from a dataset of pressure measurements which has been obtained from the same subject prior to the actual dataset. In such a case, a determined parameter determined from the actual dataset which differs with respect to the previously determined parameter shall be indicative for either an improvement or worsening depending on the previous status of the disease or a symptom accompanying it and the kind of activity represented by the parameter. The skilled person knows based on the kind of activity and previous parameter how the said parameter can be used as a reference.

Comparing the determined at least one parameter to a reference can be achieved by an automated comparison algorithm implemented on a data processing device such as a computer. Compared to each other are the values of a determined parameter and a reference for said determined parameter as specified elsewhere herein in detail. As a result of the comparison, it can be assessed whether the determined parameter is identical or differs from or is in a certain relation to the reference (e.g., is larger or lower than the reference). Based on said assessment, the subject can be identified as suffering from the muscular disability and, in particular, SMA (“rule-in”), or not (“rule-out”). For the assessment, the kind of reference will be taken into account as described elsewhere in connection with suitable references according to the invention.

Moreover, by determining the degree of difference between a determined parameter and a reference, a quantitative assessment of the muscular disability and, in particular, SMA in a subject shall be possible. It is to be understood that an improvement, worsening or unchanged overall disease condition or of symptoms thereof can be determined by comparing an actually determined parameter to an earlier determined one used as a reference. Based on quantitative differences in the value of the said parameter the improvement, worsening or unchanged condition can be determined and, optionally, also quantified. If other references, such as references from subjects with SMA are used, it will be understood that the quantitative differences are meaningful if a certain disease stage can be allocated to the reference collective. Relative to this disease stage, worsening, improvement or unchanged disease condition can be determined in such a case and, optionally, also quantified.

The said diagnosis, e.g., the assessment of the muscular disability or SMA in the subject, is indicated to the subject or another person, such as a medical practitioner or clinical analyst. Typically, this is achieved by displaying on the mobile device or the evaluation device.

Moreover, the one or more parameter can also be stored on the mobile device or indicated to the subject, typically, in real-time. The stored parameters can be assembled into a time course or similar evaluation measures. Such evaluated parameters can be provided to the subject as a feedback for activity capabilities investigated in accordance with the method of the invention. Typically, such a feedback can be provided in electronic format on a suitable display of the mobile device and can be linked to a recommendation for therapy as specified above or rehabilitation measures.

Further, the evaluated parameters can also be provided to medical practitioners in doctor's offices or hospitals as well as to other health care providers, such as, developers of diagnostic tests or drug developers in the context of clinical trials, health insurance providers or other stakeholders of the public or private health care system.

Illustratively, the method of the present invention for assessing SMA in a subject can be carried out as follows:

First, at least one parameter is determined from an existing dataset of sensor measurements obtained from said subject using a mobile device. Said dataset can have been transmitted from the mobile device to an evaluating device, such as a computer, or can be processed in the mobile device in order to derive the at least one parameter from the dataset.

Second, the determined at least one parameter is compared to a reference by, e.g., using a computer-implemented comparison algorithm carried out by the data processor of the mobile device or by the evaluating device, e.g., the computer. The result of the comparison is assessed with respect to the reference used in the comparison and based on the said assessment the subject will be identified as a subject suffering from SMA, or not.

Third, the said diagnosis, i.e. the identification of the subject as being a subject suffering from SMA, or not, is indicated to the subject or other person, such as a medical practitioner. However, it will be understood that for a final clinical diagnosis or assessment further factors or parameters can be taken into account by the clinician.

The term “identification” as used herein refers to assessing whether a subject suffers from SMA with a certain likelihood. It will be understood that the assessment can, thus, not be correct for all. However, it is typically envisaged that a statistically significant portion of the investigated subjects can be assessed, i.e. identified as suffering from SMA. How statistical significance can be determined is described elsewhere herein. Identification as used herein refers, typically, to the provision of a hint rather to a final conclusion.

Yet as an alternative or in addition, the at least one parameter underlying the diagnosis will be stored on the mobile device. Typically, it shall be evaluated together with other stored parameters by suitable evaluation tools, such as time course assembling algorithms, implemented on the mobile device which can assist electronically rehabilitation or therapy recommendation as specified elsewhere herein.

Advantageously, it has been found in the studies underlying the present invention that parameters obtained from datasets of sensor measurements in SMA patients can be used as digital biomarkers for assessing SMA in those patients, i.e. identifying those patients which suffer from SMA. The said datasets can be acquired from the SMA patients in a convenient manner by using mobile devices such as smartphones, portable multimedia devices or tablet computers on which the subjects perform active or passive pressure tests. In particular, it was found in the studies underlying the present invention that even datasets obtained by passive pressure measurements performed during other activities carried out on a smartphone are of sufficient quality for a meaningful assessment of SMA patients. The datasets acquired can be subsequently evaluated by the method of the invention for the parameter suitable as digital biomarker. Said evaluation can be carried out on the same mobile device or it can be carried out on a separate remote device. Moreover, by using such mobile devices, recommendations on life style or therapy can be provided to the patients directly, i.e., without the consultation of a medical practitioner in a doctor's office or hospital ambulance. Thanks to the present invention, the life conditions of SMA patients can be adjusted more precisely to the actual disease status due to the use of actual determined parameters by the method of the invention. Thereby, drug treatments can be selected that are more efficient or dosage regimens can be adapted to the current status of the patient. It is to be understood that the method of the invention is, typically, a data evaluation method which requires an existing dataset of activity measurements from a subject. Within this dataset, the method determines at least one parameter which can be used for assessing SMA, i.e., which can be used as a digital biomarker for SMA. Moreover, it will be understood that the method of the present invention using parameters from datasets of pressure measurements can also be applied for the assessment of muscular disabilities other than SMA. For such assessments the same principles shall apply as for SMA.

Accordingly, the method of the present invention can be used for:
assessing the disease condition;
monitoring patients in real life,
monitoring patients in on a daily basis;
investigating drug efficacy, in particular during clinical trials;
facilitating and/or aiding therapeutic decision making;

The explanations and definitions for the terms made above apply mutatis mutandis to the embodiments described herein below.

The present invention also contemplates a computer program, computer program product or computer readable storage medium having tangibly embedded said computer program, wherein the computer program comprises instructions when run on a data processing device or computer carry out the method of the present invention as specified above. Specifically, the present disclosure further encompasses:
A computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer script, wherein the computer program is adapted to perform the method according to one of the embodiments described in this description while the program is being executed on a computer,
computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network,
a computer program product having program code means, wherein the program code means can be stored or are stored on a storage medium, for performing the method according to one of the embodiments described in this description, if the program code means are executed on a computer or on a computer network,
a data stream signal, typically encrypted, comprising a dataset of pressure measurements obtained from the subject using a mobile, and
a data stream signal, typically encrypted, comprising the at least one parameter derived from the dataset of pressure measurements obtained from the subject using a mobile.

A system comprising a mobile device comprising at least one sensor and a remote device comprising a processor and a database as well as software which is tangibly embedded to said device and, when running on said device, carries out any of the methods of the invention, wherein said mobile device and said remote device are operatively linked to each other.

Under “operatively linked to each other” it is to be understood that the devices are connected as to allow data transfer from one device to the other device. Typically, it is envisaged that at least the mobile device which acquires data from the subject is connect to the remote device carrying out the steps of the methods of the invention such that the acquired data can be transmitted for processing to the remote device. However, the remote device can also transmit data to the mobile device such as signals controlling or supervising its proper function. The connection between the mobile device and the remote device can be achieved by a permanent or temporary physical connection, such as coaxial, fiber, fiber-optic or twisted-pair, 10 BASE-T cables. Alternatively, it can be achieved by a temporary or permanent wireless connection using, e.g., radio waves, such as but not limited to Wi-Fi, cellular, 3G, 4G, LTE, LTE-advanced, 5G, Bluetooth, and the like. Further details can be found elsewhere in this specification. For data acquisition, the mobile device can comprise a user interface such as screen or other equipment for data acquisition. Typically, the activity measurements can be performed on a screen comprised by a mobile device, wherein it will be understood that the said screen can have different sizes including, e.g., a 5.1 inch screen

### BRIEF DESCRIPTION OF THE DRAWINGS

FIG. 1A and FIG. 1B depict the illustrative screenshots and progression for a diagnostic test according to one or more illustrative aspects described herein. The user needs to select “Start” to begin the task.

FIG. 2 are plots illustrating various sensor feature results according to the diagnostic test depicted in FIG. 1A and FIG. 1B. Sensor feature (duration of the longest “aaah” in the test in seconds) results are in agreement with clinical anchor (forced volume vital capacity) in both studies.

FIG. 3A, FIG. 3B, and FIG. 3C depict the illustrative screenshots and progression for a diagnostic test according to one or more illustrative aspects described herein. The user needs to select “Start” to begin the task.

FIG. 4 are plots illustrating the sensor feature results according to the example 2 “Tap the monster” diagnostic test depicted in FIG. 3A, FIG. 3B, and FIG. 3C. Sensor feature (median time to hit the monster) results are in agreement with clinical anchor (go round the edge of a CD without compensatory movements) in both studies.

FIG. 5A and FIG. 5B depict the illustrative screenshots and progression for a diagnostic test according to one or more illustrative aspects described herein. The user needs to select “Start” to begin with the task.

FIG. 6 are plots illustrating the sensor feature results according to the example 3 “Squeeze the tomato”, diagnostic test depicted in FIG. 5A and FIG. 5B. Sensor feature (time difference between fingers touching the screen in seconds) results are in agreement with clinical anchor (mean of MFM004, MFM017, MFM018, MFM019,MFM020,MFM021,MFM022) in both studies.

FIG. 7A, FIG. 7B, FIG. 7C, FIG. 7D, and FIG. 7E depict the illustrative screenshots and progression for a diagnostic test according to one or more illustrative aspects described herein. The user needs to select “Start” to begin with the task.

FIG. 8 are plots illustrating the sensor feature results according to the example 4 “Walk the trail”, diagnostic test depicted in FIG. 7A, FIG. 7B, FIG. 7C, FIG. 7D, and FIG. 7E. Sensor feature (duration of drawing a shape in seconds) results are in agreement with clinical anchor (pick up 10 coins with one hand in 20 seconds) in both studies.

FIG. 9A, FIG. 9B, and FIG. 9C depict the illustrative screenshots and progression for a diagnostic test according to one or more illustrative aspects described herein. The user needs to select “Start” to begin with the task.

FIG. 10 are plots illustrating the sensor feature results according to the example 5 “Turn the phone”, diagnostic test depicted in FIG. 9A, FIG. 9B, and FIG. 9C. Sensor feature (duration of turning the phone in seconds) results are in agreement with clinical anchor (duration of pick up tennis ball, then turn hand) in both studies.

FIG. 11A and FIG. 11B depict the illustrative screenshots and progression for a diagnostic test according to one or more illustrative aspects described herein. The user needs to select “Start” to begin with the task.

FIG. 12 are plots illustrating the sensor feature results according to the example 6 “Walk the rope”, diagnostic test depicted in FIG. 11A and FIG. 11B. Sensor feature (standard deviation of acceleration magnitude to wind reaction) results are in agreement with clinical anchor (MFM32) in both studies.

FIG. 13A, FIG. 13B, and FIG. 13C depict the illustrative screenshots and progression for a diagnostic test according to one or more illustrative aspects described herein. The user needs to select “Start” to begin with the task.

FIG. 14 are plots illustrating the sensor feature results according to the example 7 “Collect the coins”, diagnostic test depicted in FIG. 13A, FIG. 13B, and FIG. 13C. Sensor feature (number of coins collected in 30 seconds) results are in agreement with clinical anchor (pick up tennis ball, then turn hand) in both studies.

FIG. 15A, FIG. 15B, and FIG. 15C depict the illustrative screenshots and progression for a diagnostic test according to one or more illustrative aspects described herein. The user needs to select “Start” to begin with the task.

FIG. 16 are plots illustrating the sensor feature results according to the example 8 “Ring the bell”, diagnostic test depicted in FIG. 15A, FIG. 15B, and FIG. 15C. Sensor feature (mean touch pressure over 10s) results are in agreement with clinical anchor (pick up 10 coins with one hand in 20 seconds) in both studies.

FIG. 17A, FIG. 17B, and FIG. 17C are plots comparing 5 different machine learning (ML) methods. The upper row shows results on the test set (i.e. the left out patient, as here leave-one-subject out cross-validation was applied). The y-axis in FIGS. 17B and 17C have the same units as depicted in FIG. 17A. Results have been calculated on the patients of the Oleos study. The results indicate that random forests and boosted trees models based on features from all tests have the potential to predict the MFM32 total score.

FIG. 18A, FIG. 18B, and FIG. 18C are plots comparing 5 different ML methods. The upper row shows results on the test set (i.e. the left out patient, as here leave-one-subject out cross-validation was applied). The y-axis in FIGS. 18B and 18C have the same units as depicted in FIG. 18A. Results have been calculated on the patients of the Oleos study. The results indicate linear regression and partial least squares regression have the potential to predict FVC.

FIG. 19 depicts an illustrative schematic diagram of an interconnected computing system that may be used, in whole or in part, to perform one or more illustrative aspects described herein.

FIG. 20 sets forth an example method for assessing the motor function of a muscular disability, in particular SMA based on active testing of the subject.

### EXAMPLES

Further to the above detailed description and algorithms provided for the many and various illustrative aspects described herein, the following Examples merely illustrate various embodiments. They shall not be construed in a way as to limit the scope of the invention.

Characteristics of the analyzed cohort of patients, collected in two different studies.

i) OLEOS Study (https://clinicaltrials.gov/ct2/showNCT02628743)

Participants analyzed: 20

Period for data analysis: smartphone data between last two clinical visits (176 days)

TABLE 1

| | Mean (SD) | Range |
|---|---|---|
| Age | 12.4 (4.1) [years] | 8.0 to 22.0 |
| Gender | 9 female, 11 male | |
| FVC | 1.61 (0.87) [liter] | 0.33 to 3.10 |

SD = Standard Deviation

ii) JEWELFISH Study

(https://clinicaltrials.gov/ct2/show/NCT03032172?term=BP39054)

Participants analyzed: 19

TABLE 2

| | Mean (SD) | Range |
|---|---|---|
| Age | 23.2 (17.2) [years] | 6.0 to 60.0 |
| Gender | 6 female, 13 male | |
| FVC | 2.75 (1.76) [liter] | 0.4 to 5.93 |

### Example 1

Dataset Acquisition Using a Computer Implemented Test for Determining the Lung Capacity (Test: Cheer the Monster), a Central Motor Function Test

TABLE 3

| | | Spearman | Spearman | | | | | |
|---|---|---|---|---|---|---|---|---|
| | | correlation | correlation | P-values | P-value | N | ICC | N ICC |
| feature | | OLEOS | Jewelfish | OLEOS | Jewelfish | OLEOS | OLEOS | OLEOS |
| std_F0¹ | pitch | −0.485 | −0.691 | 0.03 | 0.002 | 20 | 0.824 | 17 |
| | standard | | | | | | | |
| | deviation | | | | | | | |
| cv_HNR¹ | Coefficient | −0.451 | −0.574 | 0.046 | 0.016 | 20 | 0.9754 | 17 |
| | of | | | | | | | |
| | variation | | | | | | | |
| | of the | | | | | | | |
| | harmonics- | | | | | | | |
| | to-noise | | | | | | | |
| | ratio | | | | | | | |

Covariate:
¹FVC in liters,
ICC = Intraclass Correlation Coefficient

A test for measuring lung volume was implemented on a mobile phone (iPhone); see

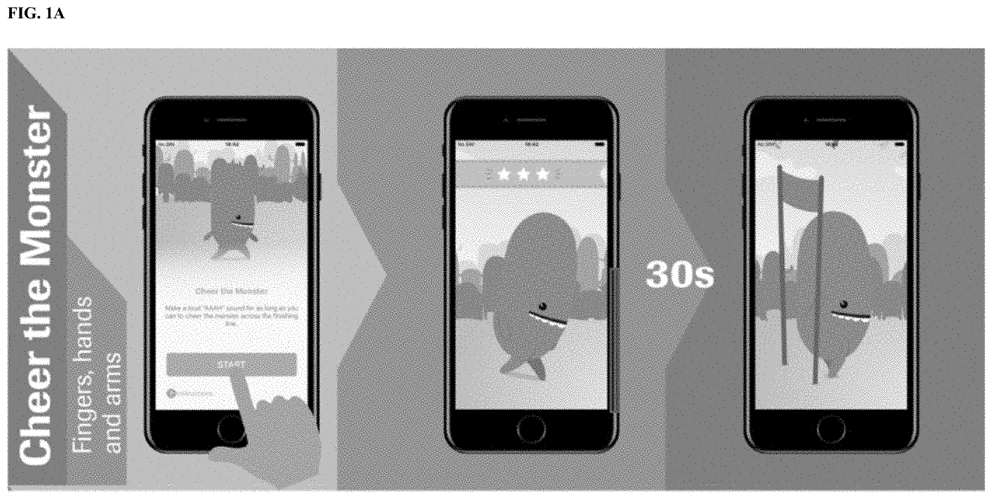

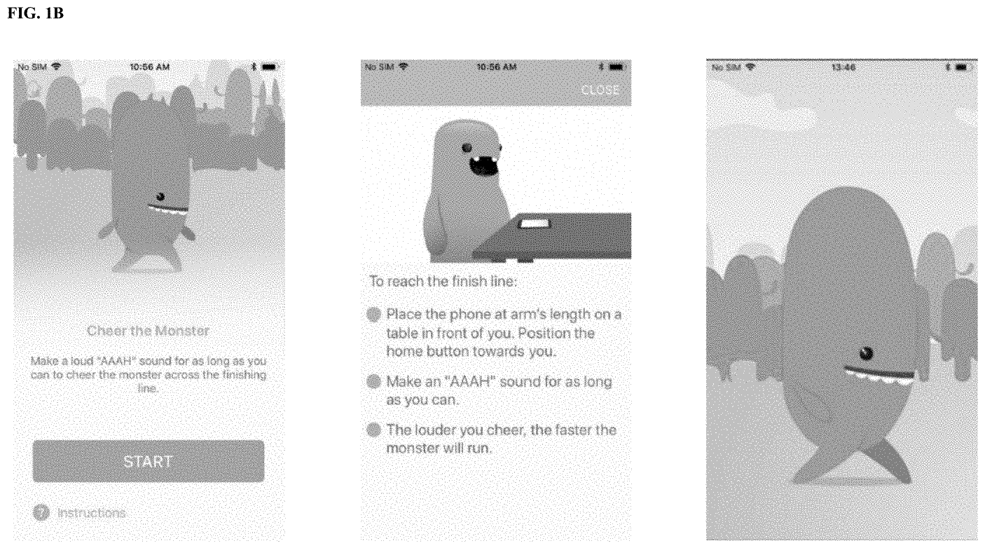

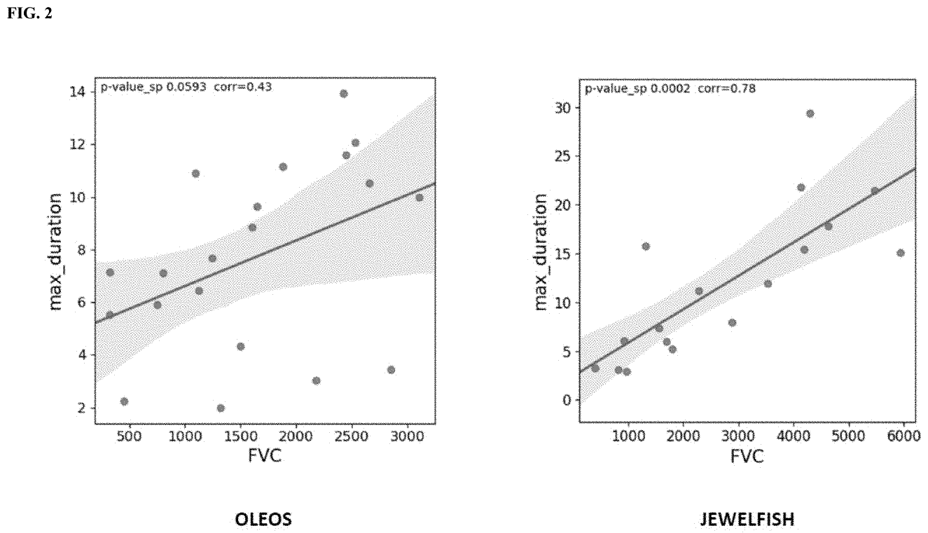

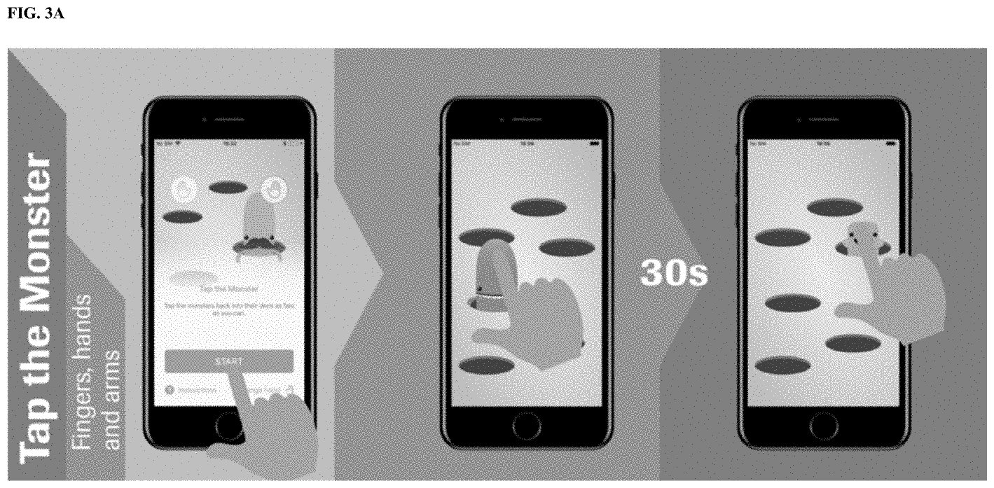

FIG. 1-2. The patients shall make a loud “aaah” sound such that the monster will reach the finish line in 30 seconds. The phone needs to be placed at arm's length on the table in front of the patient. The louder the “aaah” sound, the faster the monster run. A voice detector was used that is detecting the sustained phonation and is segmenting it each time there is a stop of ‘aahh’. The patient needs to play a game aiming to obtain maximum duration of the tone. The results of the test are expressed as said maximum duration in seconds. The standard pitch variability was determined.

The x-axis in FIG. 2 shows the correlation of the forced volume vital capacity (FVC) in milliliters and the results from the cheer the monster test. The sensor feature results are in agreement with the clinical anchor (FCV) in both studies.

### Example 2

Dataset Acquisition Using a Computer-Implemented Test for Determining Finger Strength by Pressure Measurement (Test: Tap the Monster), a Central Motor Function Test

TABLE 4

| | | Spearman | Spearman | | | | | |
|---|---|---|---|---|---|---|---|---|
| | | correlation | correlation | P-values | P-value | N | ICC | N ICC |
| feature | | OLEOS | Jewelfish | OLEOS | Jewelfish | OLEOS | OLEOS | OLEOS |
| max_pressure_std¹ | Standard | 0.474 | 0.745 | 0.035 | 0 | 20 | 0.8865 | 16 |
| | deviation | | | | | | | |
| | of | | | | | | | |
| | maximal | | | | | | | |
| | pressure | | | | | | | |
| | during tap | | | | | | | |
| max_pressure_50%¹ | Median of | 0.494 | 0.7225 | 0.027 | 0 | 20 | 0.762 | 16 |
| | maximal | | | | | | | |
| | pressure | | | | | | | |
| max_pressure_max¹ | Maximum | 0.4764 | 0.6885 | 0.034 | 0.001 | 20 | 0.889 | 16 |
| | pressure | | | | | | | |
| | tap | | | | | | | |
| time_to_hit_50%² | Median | −0.554 | −0.6075 | 0.011 | 0.006 | 20 | 0.916 | 16 |
| | time to hit | | | | | | | |
| | monster | | | | | | | |
| num_hit² | Number of | 0.463 | 0.5395 | 0.04 | 0.017 | 20 | 0.917 | 16 |
| | monster | | | | | | | |
| | hits | | | | | | | |

Covariate:
¹MFM-18,
²MFM_D3

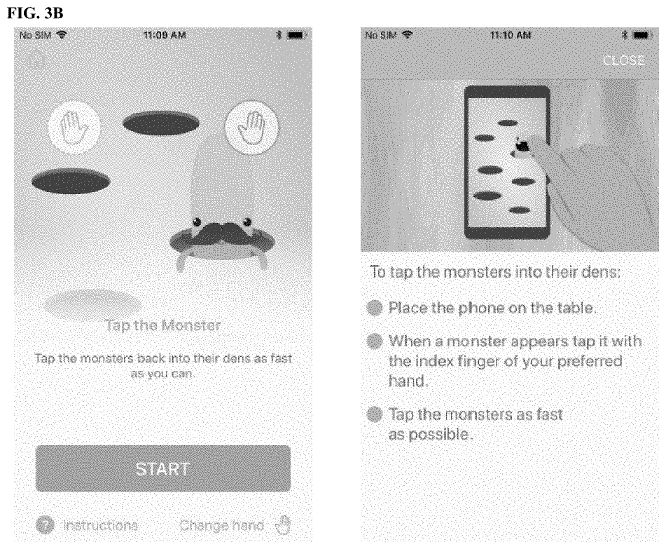

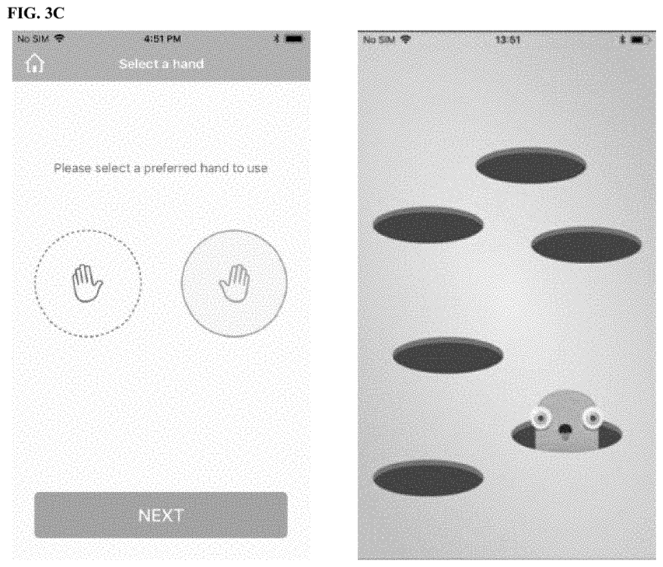

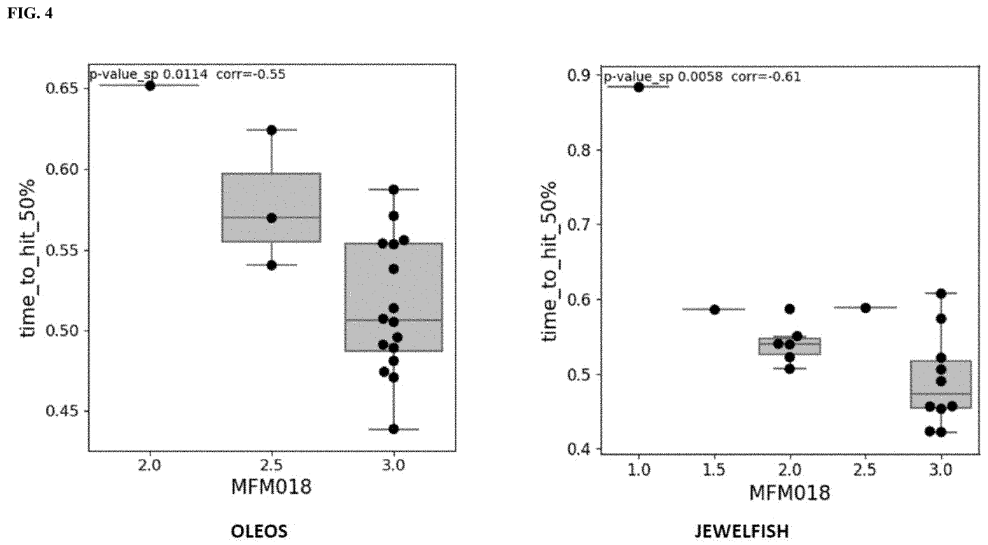

A test for pressure measuring of finger strength by pressure measurement was implemented on a mobile phone (iPhone); see FIG. 3-4. The patients shall tap the monster with the index finger such that the monsters go back into their dens. The phone should be placed on a table. The monsters should be tapped as fast as possible. The patient must select the preferred hand to use. The patient needs to play a game for 30 seconds aiming to obtain the maximum pressure of a single tap, the minimum time to tap a monster after its appearance, as well as the total number of monsters tapped within the time period of 30 seconds. The standard deviation of maximal pressure, the median of maximal pressure, the maximum pressure of a single tap, the median time to hit a monster after its appearance as well as the total numbers of monster hits obtained within 30 seconds were determined. True monster hits were protocoled events by the test. This data is transferred and the monster hitting timestamps used to calculate the median time to hit the monster.

FIG. 4 shows the correlation of the clinical anchor test and the results from the tap the monster test (time to hit 50%). The sensor feature results are in agreement with the clinical anchor (go around the edge of a CD with a finger) in both studies.

### Example 3

Dataset Acquisition Using a Computer-Implemented Test for Determining Synchronicity of 2 Fingers (Thumb and Index Finger of the Same Hand) by Measuring the Lag Time Between First and Second Fingers Touch the Screen for all Double Contacts Detected (Test: Squeeze the Tomato), a Distal Motor Function Test

TABLE 5

| | | Spearman | Spearman | | | | |
|---|---|---|---|---|---|---|---|
| | | correlation | correlation | P-values | P-value | N | ICC |
| feature | | OLEOS | Jewelfish | OLEOS | Jewelfish | OLEOS | OLEOS |
| DTA² | double touch | −0.751 | −0.877 | 0 | 0 | 19 | 0.848 |
| | asynchronicity | | | | | | |
| DTA_0_15² | double touch | −0.736 | −0.877 | 0 | 0 | 19 | 0.841 |
| | asynchronicity in | | | | | | |
| | first15 s | | | | | | |
| DTA_S² | Double touching | −0.726 | −0.882 | 0 | 0 | 19 | 0.838 |
| | asynchrony at | | | | | | |
| | successful | | | | | | |
| | pinchings | | | | | | |
| P_GAP_S² | Pinching gap | −0.505 | −0.858 | 0.027 | 0 | 19 | 0.748 |
| | time at | | | | | | |
| | successful | | | | | | |
| | pinchings | | | | | | |
| DTA¹ | double touch | −0.483 | −0.8138 | 0.036 | 0 | 19 | 0.848 |
| | asynchronicity | | | | | | |
| DTA_0 _15³ | double touch | −0.652 | −0.812 | 0.002 | 0 | 19 | 0.841 |
| | asynchronicity in | | | | | | |
| | first15 s | | | | | | |
| DTA³ | double touch | −0.657 | −0.804 | 0.002 | 0 | 19 | 0.848 |
| | asynchronicity | | | | | | |
| DTA_S³ | Double touching | −0.620 | −0.8 | 0.005 | 0 | 19 | 0.838 |
| | asynchrony at | | | | | | |
| | successful | | | | | | |
| | pinchings | | | | | | |
| SUM_P² | Total number of | 0.532 | 0.783 | 0.019 | 0 | 19 | 0.801 |
| | pinching | | | | | | |
| DTA_S¹ | Double touching | −0.498 | −0.797 | 0.03 | 0 | 19 | 0.838 |
| | asynchrony at | | | | | | |
| | successful | | | | | | |
| | pinchings | | | | | | |
| DTA_15_30² | Double touching | −0.716 | −0.789 | 0.001 | 0 | 19 | 0.853 |
| | asynchrony at | | | | | | |
| | time 15-30 sec | | | | | | |
| DTA_F² | Double touching | −0.642 | −0.768 | 0.003 | 0 | 19 | 0.785 |
| | asynchrony at | | | | | | |
| | failed pinchings | | | | | | |
| DTA_F³ | Double touching | −0.580 | −0.738 | 0.009 | 0.001 | 19 | 0.785 |
| | asynchrony at | | | | | | |
| | failed pinchings | | | | | | |
| DTA_15_30¹ | Double touching | −0.456 | −0.745 | 0.049 | 0.001 | 19 | 0.853 |
| | asynchrony at | | | | | | |
| | time 15-30 sec | | | | | | |
| DTA_0_15⁴ | double touch | −0.485 | −0.681 | 0.035 | 0.003 | 19 | 0.841 |
| | asynchronicity in | | | | | | |
| | first 15 s | | | | | | |
| DTA⁴ | | −0.546 | −0.674 | 0.016 | 0.003 | 19 | 0.848 |
| DTA_15_30³ | | −0.634 | −0.688 | 0.004 | 0.003 | 19 | 0.853 |
| DTA_S⁴ | | −0.586 | −0.649 | 0.008 | 0.006 | 19 | 0.838 |
| DTA_15_30⁴ | | −0.541 | −0.583 | 0.017 | 0.018 | 19 | 0.853 |
| P_TP_0_15³ | | −0.494 | 0.517 | 0.032 | 0.034 | 19 | 0.925 |

Covariate:
¹MFM-17, 18, 19, 22;
²MFM_D3;
³Total 32 = MFM total score;
⁴MFM-17
ICC: Intraclass Correlation Coefficient,
DTA: double touch asynchronicity,
P_GA: Pinching gap time

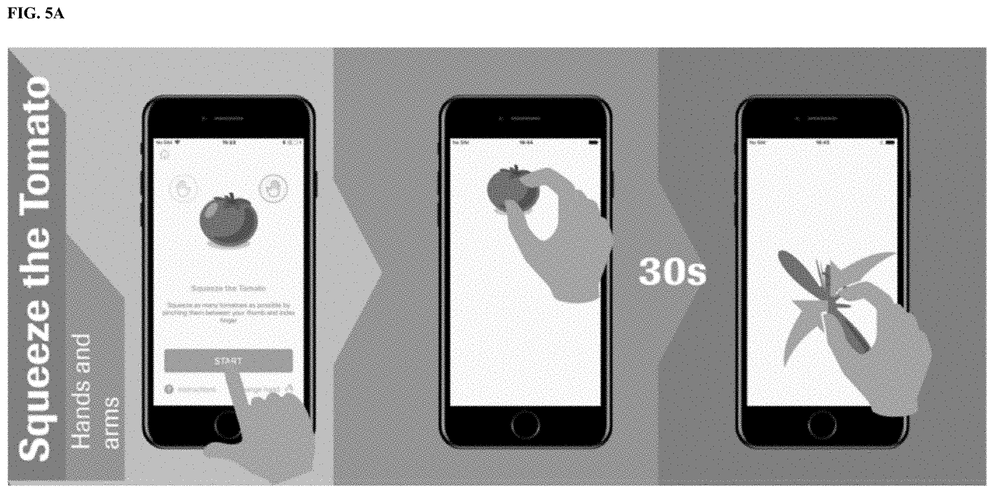

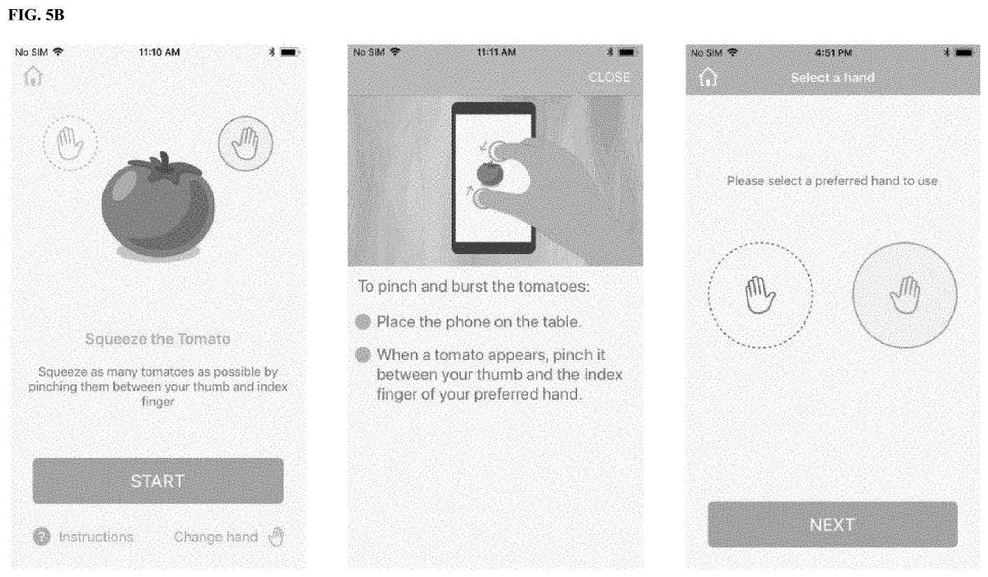

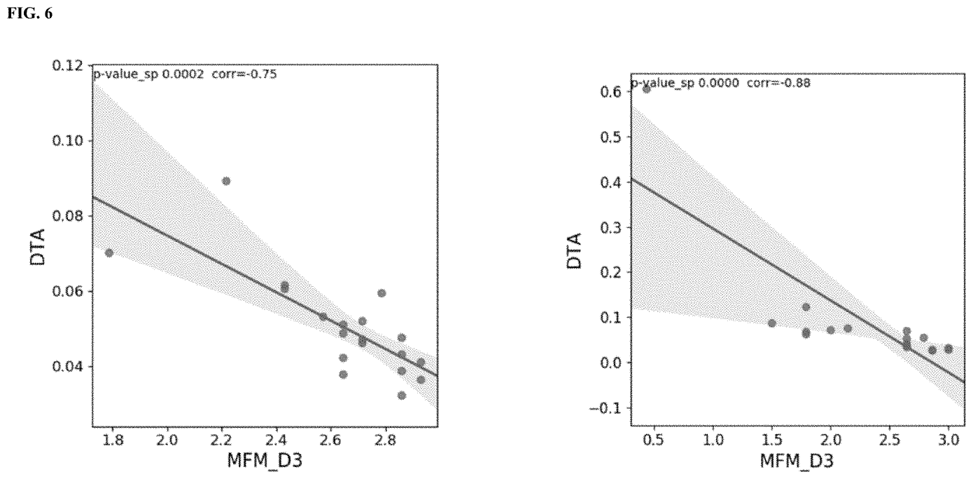

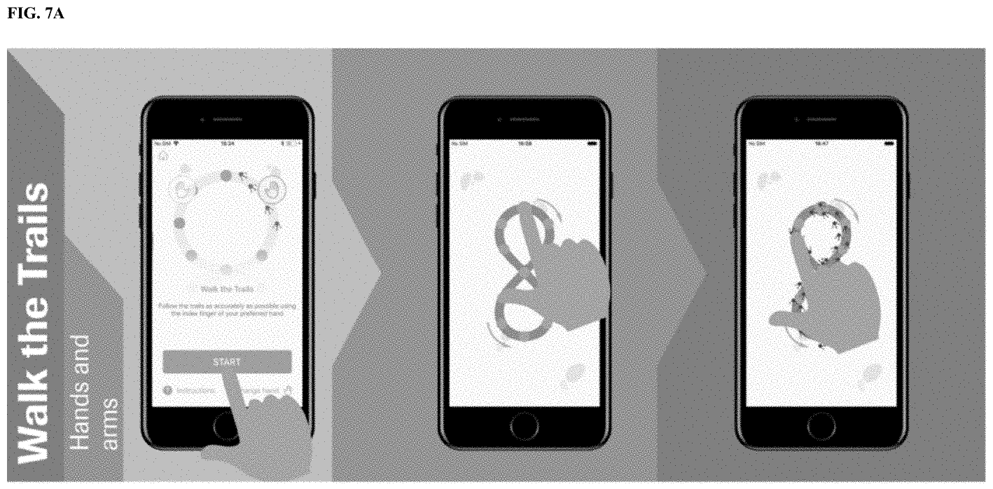

A test for double touching asynchronicity (DTA) was implemented on a mobile phone (iPhone); see FIG. 5-6. The patients shall squeeze as many tomatoes as possible within 30 seconds by pinching them between the thumb and index finger of the indicated hand. The phone needs to be placed on the table. The referred hand needs to be selected. The patient needs to play a game for 30 seconds.

FIG. 6 shows the correlation of the clinical anchor test and the results from the squeeze the tomato test (DTA). The sensor feature results are in agreement with the clinical anchor in both studies.

### Example 4

Dataset Acquisition Using a Computer-Implemented Test for Determine by Measuring the Time Required to Draw the FIGURE “8” (Test: Walk the Trail), a Central Motor Function Test

TABLE 6

| | | Spearman | Spearman | | | | | |
|---|---|---|---|---|---|---|---|---|
| | | correlation | correlation | P-values | P-value | N | ICC | N ICC |
| feature | | OLEOS | Jewelfish | OLEOS | Jewelfish | OLEOS | OLEOS | OLEOS |
| SQUARE_Mag_areaError¹ | The ratio of | 0.456 | 0.575 | 0.049 | 0.02 | 19 | 0.756 | 16 |
| | the area | | | | | | | |
| | under the | | | | | | | |
| | curve when | | | | | | | |
| | plotting the | | | | | | | |
| | x-y drawing | | | | | | | |
| | data points | | | | | | | |
| | in polar | | | | | | | |
| | coordinates | | | | | | | |
| | (normalized | | | | | | | |
| | to the | | | | | | | |
| | number of | | | | | | | |
| | data points) | | | | | | | |
| | to those of | | | | | | | |
| | the | | | | | | | |
| | interpolated | | | | | | | |
| | reference | | | | | | | |
| | coordinates. | | | | | | | |
| SQUARE_areaError¹ | Area of | 0.456 | 0.575 | 0.049 | 0.02 | 19 | 0.756 | 16 |
| | deviation | | | | | | | |
| | between | | | | | | | |
| | drawn | | | | | | | |
| | square and | | | | | | | |
| | interpolated | | | | | | | |
| | reference | | | | | | | |
| | coordinates | | | | | | | |
| SQUARE_sqrtError² | calculated as | 0.467 | 0.537 | 0.044 | 0.032 | 19 | 0.8296 | 16 |
| | the square | | | | | | | |
| | root of the | | | | | | | |
| | error | | | | | | | |
| | between the | | | | | | | |
| | AUC of the | | | | | | | |
| | shape drawn | | | | | | | |
| | versus the | | | | | | | |
| | reference | | | | | | | |
| | points using | | | | | | | |
| | the | | | | | | | |
| | trapezoidal | | | | | | | |
| | rule for | | | | | | | |
| | integration. | | | | | | | |
| | This feature | | | | | | | |
| | is also | | | | | | | |
| | normalized | | | | | | | |
| | by the | | | | | | | |
| | number of | | | | | | | |
| | touch data | | | | | | | |
| | points drawn | | | | | | | |

Covariate:
¹MFM-17, 18, 19, 22;
²MFM-19
ICC: Intraclass Correlation Coefficient

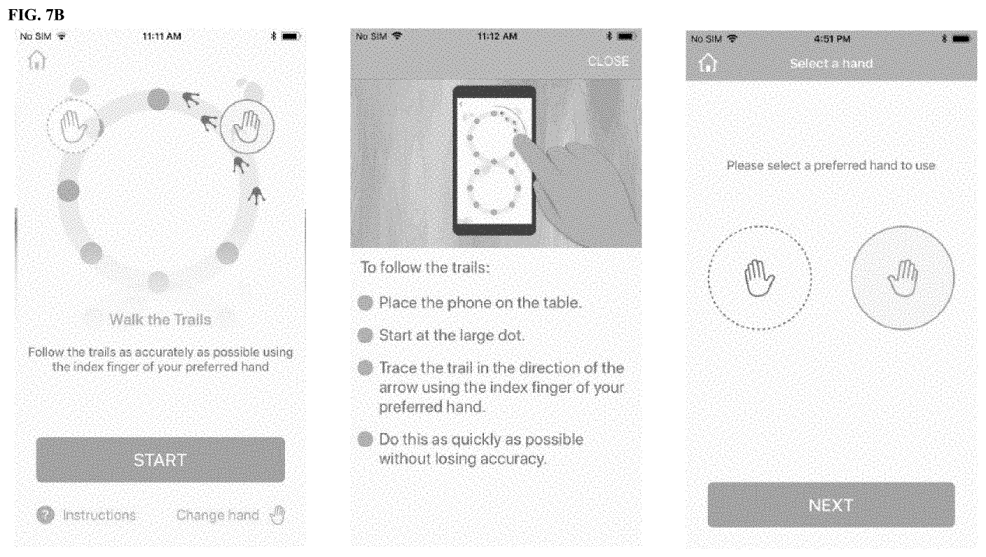

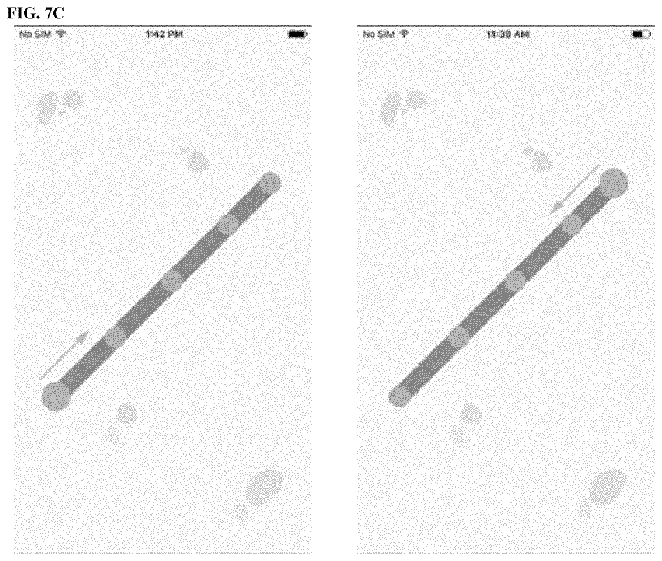

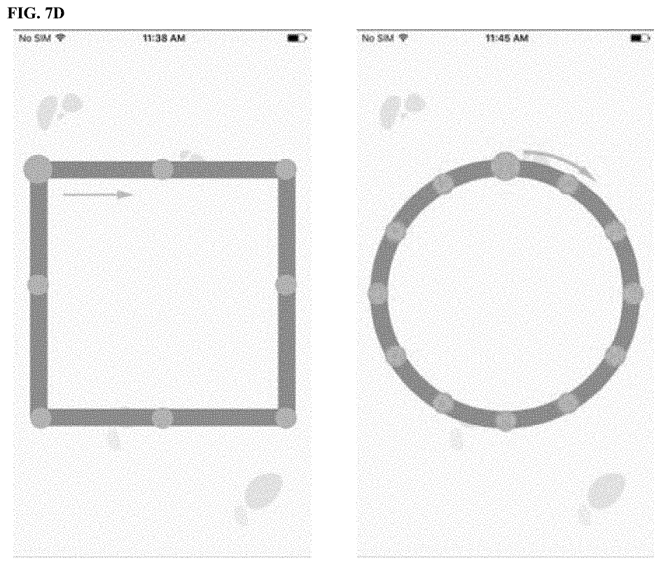

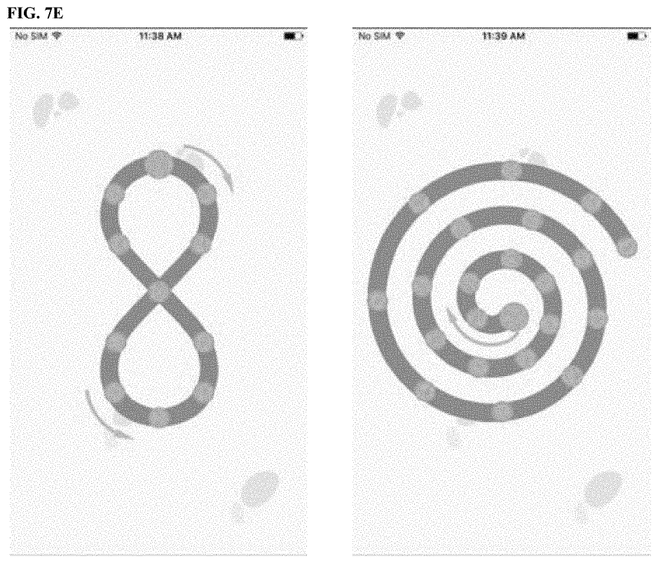

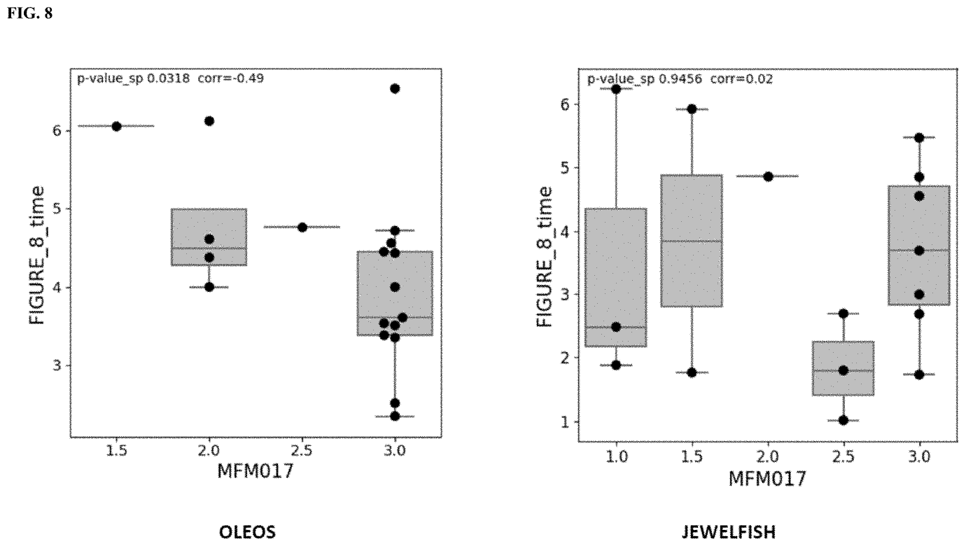

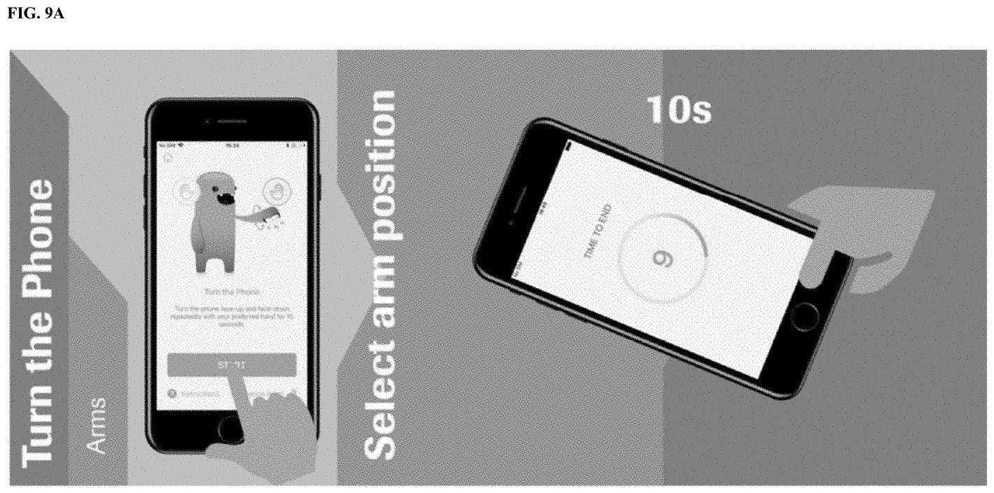

A test for was implemented on a mobile phone (iPhone); see FIG. 7-8. The patients shall follow a shape as accurately as possible using the index finger of the preferred hand. The phone should be placed on the table. The preferred hand should be selected. The patient should start at the largest dot. One of the shapes is the number “8”. One of the shapes is a stick. One of the shapes is a square. One of the shapes is a circle. One of the shapes is a spiral. The patient needs to play a game for 30 seconds and follow the shape as quickly as possible without losing accuracy.

FIG. 8 shows the correlation of the clinical anchor test and the results from the walk the trail test (draw an “8” time). The sensor feature results are not in clear association with the clinical anchor (pick up 10 coins with one hand in 20 seconds) in both studies.

### Example 5

Dataset Acquisition Using a Computer-Implemented Test for Determining by Measuring the Time Required to Turn the Phone (Test: Turn the Phone), an Axial Motor Function Test

TABLE 7

| | | Spearman | Spearman | | | | | |
|---|---|---|---|---|---|---|---|---|
| | | correlation | correlation | P-values | P-value | N | ICC | N ICC |
| feature | | OLEOS | Jewelfish | OLEOS | Jewelfish | OLEOS | OLEOS | OLEOS |
| num_turns | Number | 0.537 | 0.697 | 0.048 | 0.002 | 14 | 0.959 | 12 |
| | of turns | | | | | | | |
| speed_median | Average | 0.631 | 0.624 | 0.016 | 0.01 | 14 | 0.946 | 12 |
| | turn | | | | | | | |
| | speed | | | | | | | |
| speed_max | Maximal | 0.644 | 0.615 | 0.013 | 0.011 | 14 | 0.930 | 12 |
| | turn | | | | | | | |
| | speed | | | | | | | |
| speed_max | Maximal | 0.701 | 0.582 | 0.005 | 0.018 | 14 | 0.930 | 12 |
| | turn | | | | | | | |
| | speed | | | | | | | |
| speed_max | Maximal | 0.624 | 0.565 | 0.017 | 0.023 | 14 | 0.930 | 12 |
| | turn | | | | | | | |
| | speed | | | | | | | |
| speed_max | Maximal | 0.536 | 0.555 | 0.048 | 0.026 | 14 | 0.930 | 12 |
| | turn | | | | | | | |
| | speed | | | | | | | |
| speed_median | Average | 0.613 | 0.545 | 0.02 | 0.029 | 14 | 0.946 | 12 |
| | turn | | | | | | | |
| | speed | | | | | | | |
| num_amplitude_halts | Number | 0.650 | 0.509 | 0.012 | 0.044 | 14 | 0.8776 | 12 |
| | of | | | | | | | |
| | hesitations | | | | | | | |
| speed_mad | Median | 0.587 | 0.506 | 0.027 | 0.046 | 14 | 0.9376 | 12 |
| | absolute | | | | | | | |
| | deviation | | | | | | | |
| | of speed | | | | | | | |
| speed_median | Average | 0.696 | 0.498 | 0.006 | 0.05 | 14 | 0.946 | 12 |
| | turn | | | | | | | |
| | speed | | | | | | | |

Covariate:
1: MFM_9_15_20_21 = sum of MFM scores 9, 15, 20, 21;
2: Total32 = MFM total score;
3: MFM010;
4: MFM_D2;
5: MFM021
ICC: Intraclass Correlation Coefficient

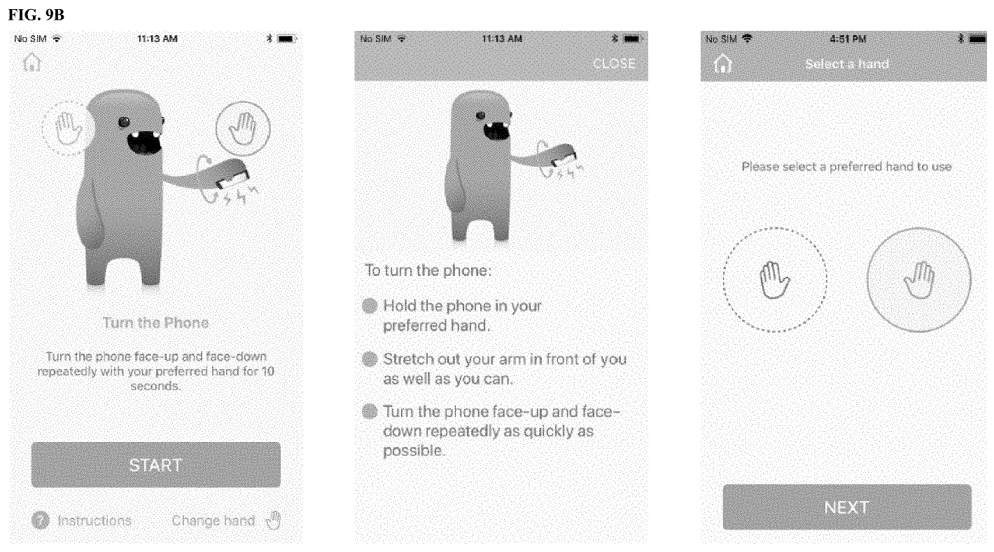

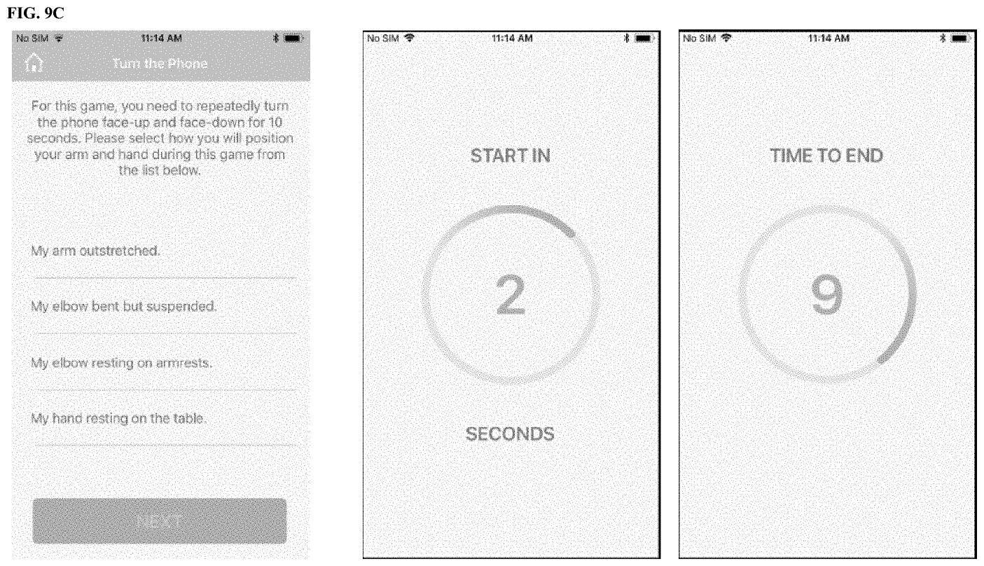

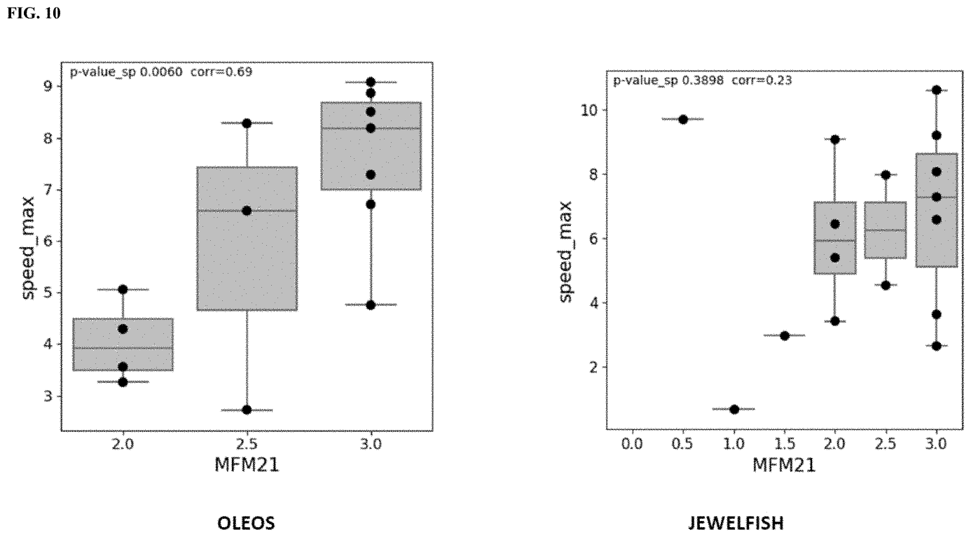

A test for was implemented on a mobile phone (iPhone); see FIG. 9-10. The patients shall turn the phone face-up and face-down repeatedly with the preferred hand for 10 seconds. The phone should be held in the preferred hand. The arm should be stretched out in front of the patient as well as possible. The patient shall indicate the position of the arm, i.e. outstretched, elbow bent but suspended, elbow resting on armrest or hand resting on table. The turn speed of a single turn as well as the number of turns in 10 seconds are measured.

FIG. 10 shows the correlation of the clinical anchor test and the results from the turn the phone test (maximum speed of a single turn in seconds). The sensor feature results are in clear association with the clinical anchor (pick up tennis ball, then turn hand) in both studies. For the clinical anchor there is no unit. It is on a scale of 0, 1, 2, 3, or 4. Values between 2 and 3 show an average in clinic measurements of two subsequent clinical visits. The selected feature is the average maximal turn speed, as measure in angular velocity (rad/s), per turn. The feature (maximum speed of a single turn in seconds was calculated based on detected and segmented turns.

### Example 6

Dataset Acquisition Using a Computer-Implemented Test for Determining by Measuring Variability of the Acceleration Occurring when Turning the Phone while Reacting/Compensating for Sudden Wind Movements (Test: Walk the Rope), an Axial Motor Function Test

TABLE 8

| | | Spearman | Spearman | | | | | |
|---|---|---|---|---|---|---|---|---|
| | | correlation | correlation | P-values | P-value | N | ICC | N ICC |
| feature | | OLEOS | Jewelfish | OLEOS | Jewelfish | OLEOS | OLEOS | OLEOS |
| reaction_acc_mag_stn | Standard | −0.593 | −0.785 | 0.025 | 0 | 14 | 0.899 | 12 |
| | deviation | | | | | | | |
| | of | | | | | | | |
| | acceleration | | | | | | | |
| | magnitude | | | | | | | |
| | to wind | | | | | | | |
| | reaction | | | | | | | |
| reaction_acc_mag_stn | Standard | −0.613 | −0.768 | 0.02 | 0.001 | 14 | 0.899 | 12 |
| | deviation | | | | | | | |
| | of | | | | | | | |
| | acceleration | | | | | | | |
| | magnitude | | | | | | | |
| | to wind | | | | | | | |
| | reaction | | | | | | | |
| acc_mag_std_0_15 | Standard | 0.637 | 0.734 | 0.014 | 0.001 | 14 | 0.825 | 12 |
| | deviation | | | | | | | |
| | of | | | | | | | |
| | acceleration | | | | | | | |
| | magnitude | | | | | | | |
| | in 0-15 s | | | | | | | |
| acc_mag_stn_0_15 | Standard | −0.637 | −0.722 | 0.014 | 0.002 | 14 | 0.909 | 12 |
| | deviation | | | | | | | |
| | of | | | | | | | |
| | acceleration | | | | | | | |
| | magnitude | | | | | | | |
| | in 0-15 s | | | | | | | |
| acc_mag_std_0_15 | Standard | 0.596 | 0.697 | 0.025 | 0.003 | 14 | 0.825 | 12 |
| | deviation | | | | | | | |
| | of | | | | | | | |
| | acceleration | | | | | | | |
| | magnitude | | | | | | | |
| | in 0-15 s | | | | | | | |
| gyr_x_std_15_30 | Gyroscop | −0.574 | −0.708 | 0.032 | 0.003 | 14 | 0.850 | 12 |
| | x-axis | | | | | | | |
| | standard | | | | | | | |
| | deviation | | | | | | | |
| | in 15-30 s | | | | | | | |
| acc_mag_stn_0_15 | Standard | −0.596 | −0.682 | 0.025 | 0.004 | 14 | 0.909 | 12 |
| | deviation | | | | | | | |
| | of | | | | | | | |
| | acceleration | | | | | | | |
| | magnitude | | | | | | | |
| | in 0-15 s | | | | | | | |
| reaction_acc_mag_std | Standard | 0.624 | 0.677 | 0.017 | 0.004 | 14 | 0.750 | 12 |
| | deviation | | | | | | | |
| | of | | | | | | | |
| | acceleration | | | | | | | |
| | magnitude | | | | | | | |
| | to wind | | | | | | | |
| | reaction | | | | | | | |
| reaction_acc_mag_std | Standard | 0.631 | 0.653 | 0.016 | 0.006 | 14 | 0.750 | 12 |
| | deviation | | | | | | | |
| | of | | | | | | | |
| | acceleration | | | | | | | |
| | magnitude | | | | | | | |
| | to wind | | | | | | | |
| | reaction | | | | | | | |
| gyr_z_stn_15_30 | Gyroscope | 0.833 | −0.620 | 0 | 0.014 | 14 | 0.705 | 12 |
| | z-axis | | | | | | | |
| | standard | | | | | | | |
| | deviation | | | | | | | |
| | in 15-30 s | | | | | | | |
| reaction_gyr_mag_median | Median | 0.713 | 0.584 | 0.004 | 0.017 | 14 | 0.708 | 12 |
| | of | | | | | | | |
| | gyroscope | | | | | | | |
| | magnitude | | | | | | | |
| | to wind | | | | | | | |
| | reaction | | | | | | | |
| acc_z_stn_0_15 | Standard | −0.661 | −0.562 | 0.01 | 0.023 | 14 | 0.891 | 12 |
| | deviation | | | | | | | |
| | of z-axis | | | | | | | |
| | accelerati | | | | | | | |
| | on in 0-15 s | | | | | | | |
| acc_z_stn_0_30 | Standard | −0.713 | −0.556 | 0.004 | 0.025 | 14 | 0.887 | 12 |
| | deviation | | | | | | | |
| | of z-axis | | | | | | | |
| | accelerati | | | | | | | |
| | on in 0-30 s | | | | | | | |
| mag_x_stn_15_30 | Standard | 0.691 | 0.521 | 0.006 | 0.047 | 14 | 0.936 | 12 |
| | deviation | | | | | | | |
| | of x-axis | | | | | | | |
| | magneto | | | | | | | |
| | meter in | | | | | | | |
| | 15-30 s | | | | | | | |
| mag_mag_stn_15_30 | Standard | −0.644 | 0.516 | 0.013 | 0.049 | 14 | 0.809 | 12 |
| | deviation | | | | | | | |
| | of | | | | | | | |
| | magnitude | | | | | | | |
| | magnetometer | | | | | | | |
| | in 15-30 s | | | | | | | |
| mag_mag_stn_15_30 | | 0.644 | −0.516 | 0.013 | 0.049 | 14 | 0.929 | 12 |

ICC: Intraclass Correlation Coefficient

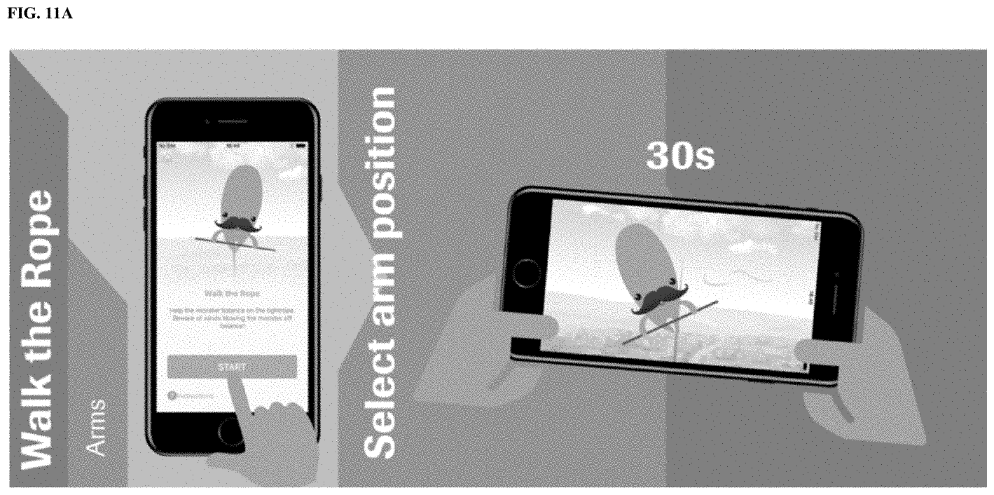

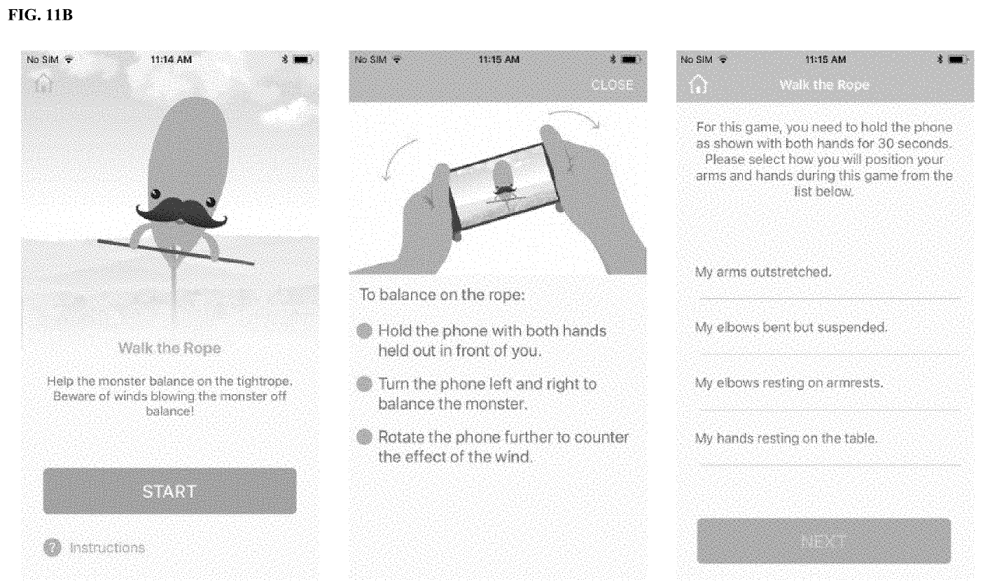

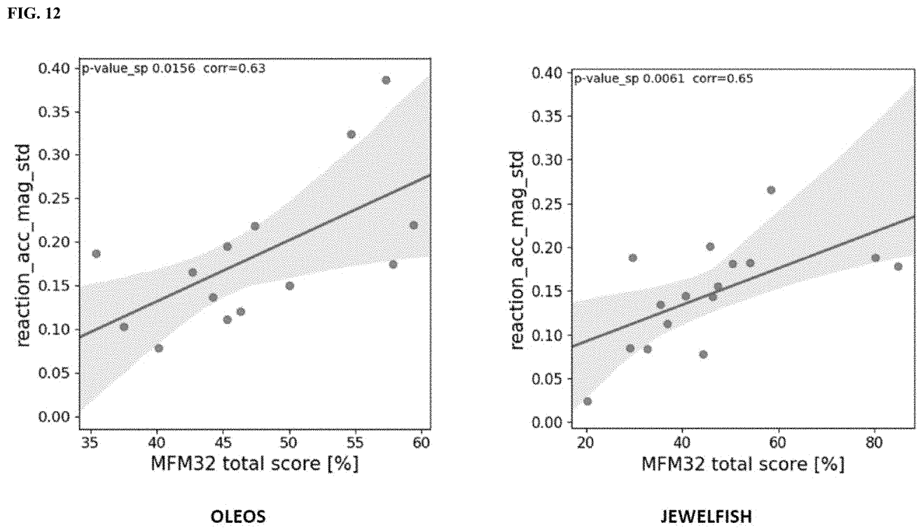

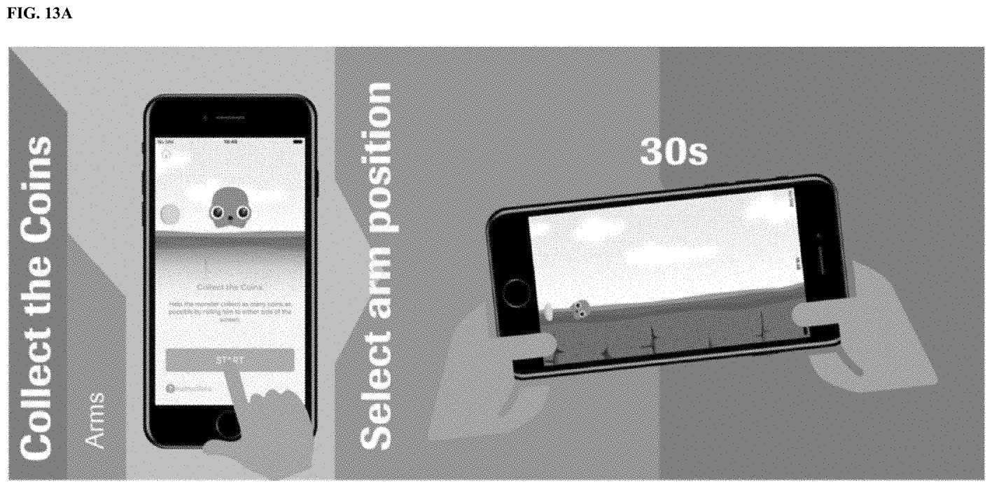

A test for was implemented on a mobile phone (iPhone); see FIG. 11-12. The patients shall balance a monster on a rope while wind is blowing the monster off balance. The phone should be held in both hands. The phone needs to be turned left and right to balance the monster. The phone can be rotated to further counter the effect of the wind. The patient shall indicate the position of the arm, i.e. outstretched, elbow bent but suspended, elbow resting on armrest or hand resting on table. The test lasts 30 seconds.

FIG. 12 shows the correlation of the clinical anchor test and the results from the walk the rope test (Standard deviation of acceleration magnitude to wind reaction in m/s²). In the test when balancing the monster, their sometimes comes a wind challenge and this is the reaction in the first 2s after that and how much variability in the hand movements is happening. This is an average over all the wind challenge in one test run. The sensor feature results are in clear association with the clinical anchor (MFM32) in both studies.

### Example 7

Dataset Acquisition Using a Computer-Implemented Test for Determining by Measuring the Number of Collected Coins in that the Patient has to Tilt the Phone Fast from Side to Side to Collect the Coins (Test: Collect the Coins), an Axial Motor Function Test

TABLE 9

| | | Spearman | Spearman | | | | | |
|---|---|---|---|---|---|---|---|---|
| | | correlation | correlation | P-values | P-value | N | ICC | N ICC |
| feature | | OLEOS | Jewelfish | OLEOS | Jewelfish | OLEOS | OLEOS | OLEOS |
| max_coin_15_30¹ | Maximal | 0.564 | 0.795 | 0.036 | 0 | 14 | 0.928 | 12 |
| | number of | | | | | | | |
| | coints in 15-30 s | | | | | | | |
| mean_gyroScalar_0_15 | Mean | 0.575 | 0.793 | 0.031 | 0 | 14 | 0.831 | 12 |
| | gyroscope | | | | | | | |
| | signal in 0-15 s | | | | | | | |
| num_collected_ coin_15_30¹ | Number of | 0.564 | 0.786 | 0.036 | 0 | 14 | 0.928 | 12 |
| | collected coins | | | | | | | |
| | in 15-30 | | | | | | | |
| time_per_coin_15_30¹ | Time per | −0.564 | −0.786 | 0.036 | 0 | 14 | 0.911 | 12 |
| | collected coin | | | | | | | |
| | in 15-30 s | | | | | | | |
| max_coin¹ | Maximal | 0.540 | 0.770 | 0.046 | 0 | 14 | 0.951 | 12 |
| | number of | | | | | | | |
| | coins | | | | | | | |
| num_collected_coin¹ | Number of | 0.540 | 0.770 | 0.046 | 0 | 14 | 0.968 | 12 |
| | collected coins | | | | | | | |
| max_coin_0_15¹ | Maximal | 0.574 | 0.726 | 0.032 | 0.001 | 14 | 0.917 | 12 |
| | number of | | | | | | | |
| | coins in 0-15 s | | | | | | | |
| time_per_coin_0_15¹ | Time per | −0.574 | −0.726 | 0.032 | 0.001 | 14 | 0.855 | 12 |
| | collected coin | | | | | | | |
| | in 0-15 s | | | | | | | |
| num_collected_coin_0_15¹ | Number of | 0.574 | 0.726 | 0.032 | 0.001 | 14 | 0.917 | 12 |
| | collected coins | | | | | | | |
| | in 0-15 s | | | | | | | |
| mean_gyro_Z_0_15² | Mean | 0.568 | 0.710 | 0.034 | 0.001 | 14 | 0.860 | 12 |
| | gyroscope z- | | | | | | | |
| | axis signal in | | | | | | | |
| | 0-15 s | | | | | | | |
| gap_time_coin_10_20¹ | Time between | −0.575 | −0.701 | 0.031 | 0.004 | 14 | 0.918 | 12 |
| | coins in 10-20 s | | | | | | | |
| gap_time_coin_0_15¹ | Time between | −0.557 | −0.671 | 0.038 | 0.004 | 14 | 0.879 | 12 |
| | coins in 0-15 s | | | | | | | |
| max_coin_0_10¹ | Maximal coins | 0.580 | 0.650 | 0.03 | 0.005 | 14 | 0.959 | 12 |
| | in 0-10 s | | | | | | | |
| num_collected_coin_0_10¹ | Number of | 0.569 | 0.650 | 0.034 | 0.005 | 14 | 0.952 | 12 |
| | collected coins | | | | | | | |
| | in 0-10 s | | | | | | | |
| time_per_coin_0_10¹ | Time per coin | −0.569 | −0.650 | 0.034 | 0.005 | 14 | 0.925 | 12 |
| | in 0-10 s | | | | | | | |
| gap_time_coin_0_10¹ | Time between | −0.588 | −0.650 | 0.027 | 0.006 | 14 | 0.876 | 12 |
| | coins in 0-10 s | | | | | | | |
| max_coin_15_30² | Maximal | 0.556 | 0.591 | 0.039 | 0.012 | 14 | 0.928 | 12 |
| | number of | | | | | | | |
| | coins in 15-30 s | | | | | | | |
| num_collected_coin_15_30² | Number of | 0.556 | 0.590 | 0.039 | 0.013 | 14 | 0.928 | 12 |
| | collected coins | | | | | | | |
| | in 15-30 | | | | | | | |
| time_per_coin_15_30² | Time per | −0.556 | −0.590 | 0.039 | 0.013 | 14 | 0.911 | 12 |
| | collected coin | | | | | | | |
| | in 15-30 s | | | | | | | |
| max_coin_10_20⁴ | Maximal | 0.604 | 0.588 | 0.022 | 0.013 | 14 | 0.867 | 12 |
| | number of | | | | | | | |
| | coins in 0-15 s | | | | | | | |
| num_collected_coin_10_20⁴ | Number of | 0.639 | 0.588 | 0.014 | 0.013 | 14 | 0.873 | 12 |
| | collected coins | | | | | | | |
| | in 10-20 s | | | | | | | |
| time_per_coin_10_20⁴ | | −0.639 | −0.588 | 0.014 | 0.013 | 14 | 0.888 | 12 |
| mean_gyroScalar_0_15⁴ | Mean | 0.604 | 0.563 | 0.022 | 0.019 | 14 | 0.831 | 12 |
| | magnitude of | | | | | | | |
| | gyroscope | | | | | | | |
| | signal in 0-15 s | | | | | | | |
| mean_gyroScalar_10_20⁴ | | 0.581 | 0.558 | 0.029 | 0.02 | 14 | 0.864 | 12 |
| time_per_coin⁴ | | −0.564 | −0.550 | 0.036 | 0.022 | 14 | 0.937 | 12 |
| max_coin⁴ | Maximal | 0.585 | 0.534 | 0.028 | 0.027 | 14 | 0.951 | 12 |
| | number of | | | | | | | |
| | coins | | | | | | | |
| num_collected_coin⁴ | Number of | 0.585 | 0.5341 | 0.028 | 0.027 | 14 | 0.968 | 12 |
| | collected coins | | | | | | | |
| gap_time_coin_15_30³ | Time between | −0.664 | −0.558 | 0.01 | 0.031 | 14 | 0.879 | 12 |
| | coins in 10-20 s | | | | | | | |
| gap_time_coin_10_20³ | Time between | −0.644 | −0.540 | 0.013 | 0.038 | 14 | 0.917 | 12 |
| | coins in 10-20 s | | | | | | | |
| max_coin_15_30⁴ | | 0.545 | 0.505 | 0.044 | 0.039 | 14 | 0.928 | 12 |
| max_coin_0_15⁴ | Maximal | 0.582 | 0.502 | 0.029 | 0.04 | 14 | 0.917 | 12 |
| | number of | | | | | | | |
| | coins in 0-15 s | | | | | | | |
| time_per_coin_0_15⁴ | | −0.582 | −0.502 | 0.029 | 0.04 | 14 | 0.855 | 12 |
| num_collected_coin_0_15⁴ | Number of | 0.582 | 0.501517962 | 0.029 | 0.04 | 14 | 0.917 | 12 |
| | collected coins | | | | | | | |
| | in 0-15 s | | | | | | | |
| num_collected_coin_15_30⁴ | Number of | 0.545 | 0.495 | 0.044 | 0.044 | 14 | 0.928 | 12 |
| | collected coins | | | | | | | |
| | in 15-30 | | | | | | | |
| time_per_coin_15_30⁴ | Time between | −0.545 | −0.495 | 0.044 | 0.044 | 14 | 0.911 | 12 |
| | coins in 15-20 s | | | | | | | |
| mean_gyroScalar_0_10⁴ | | 0.604 | 0.494 | 0.022 | 0.044 | 14 | 0.770 | 12 |
| gap_time_coin³ | Time between | −0.678 | −0.508 | 0.008 | 0.045 | 14 | 0.922 | 12 |
| | coin | | | | | | | |

Covariate:
¹MFM_9_15_20_21 = sum of MFM 9, 15, 20, 21;
²MFM9;
³AGEIC;
⁴MFM21;
5: MFM015
ICC: Intraclass Correlation Coefficient

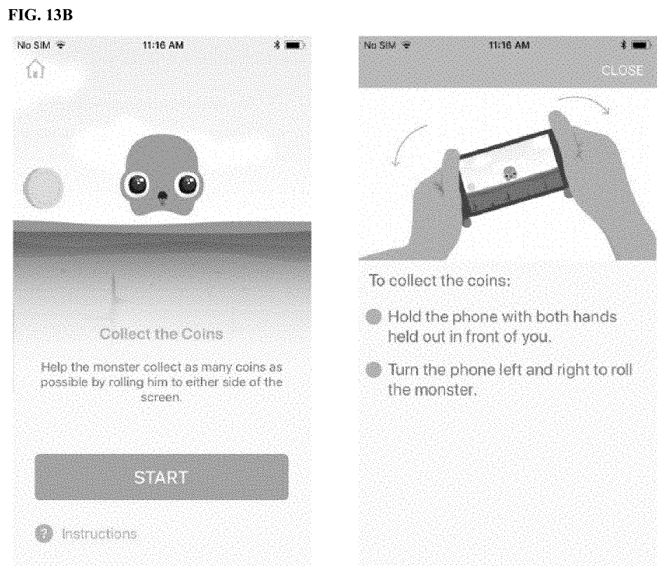

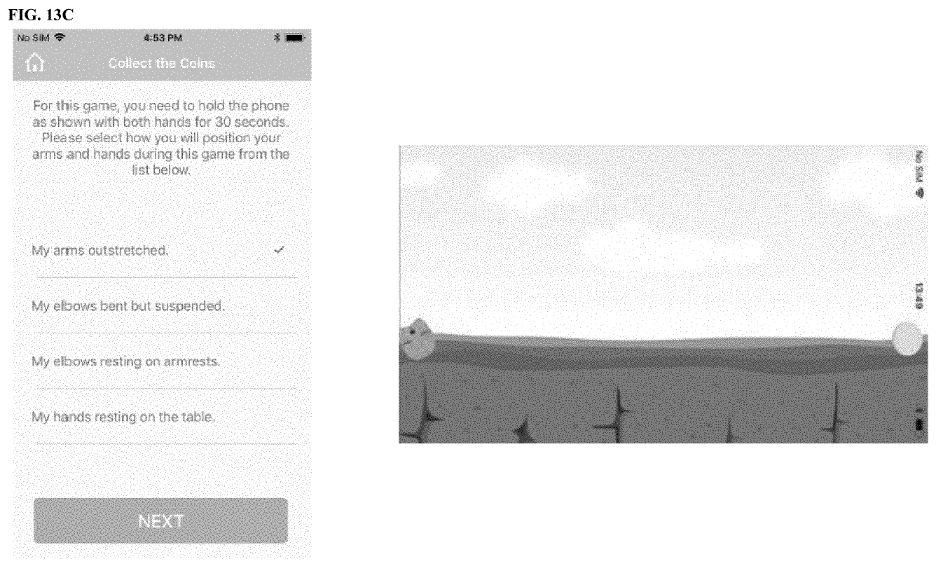

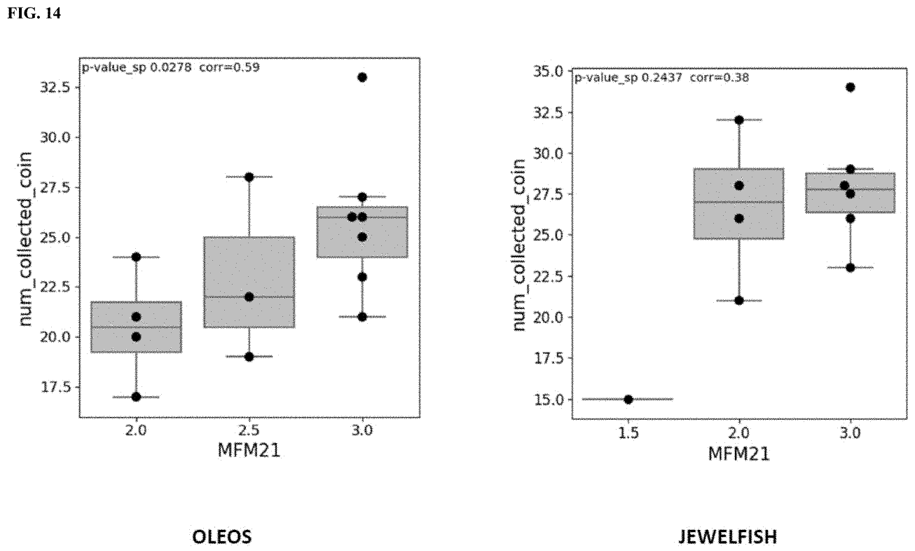

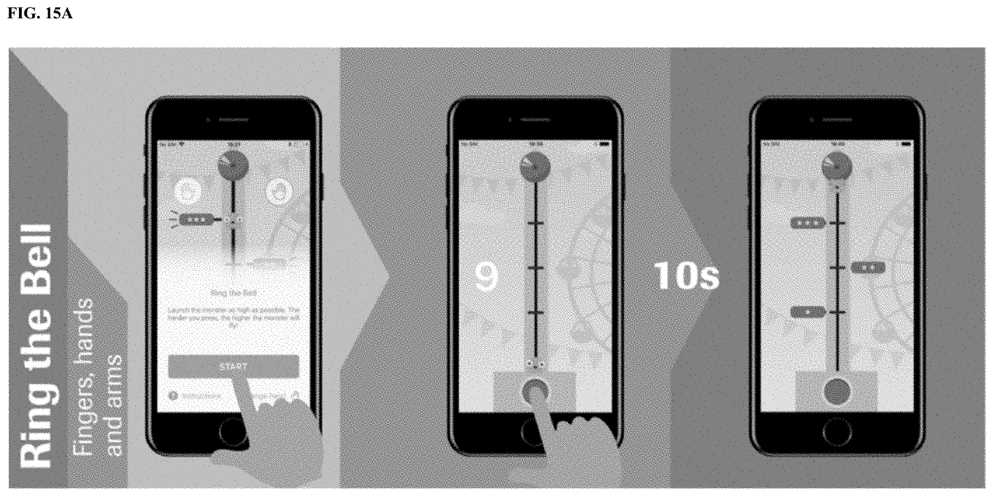

A test for was implemented on a mobile phone (iPhone); see FIG. 13-14. The phone should be held in both hands. The patients shall tilt the phone fast from side to side and thus collect as many coins as possible. The patient shall indicate the position of the arm, i.e. outstretched, elbow bent but suspended, elbow resting on armrest or hand resting on table. The test lasts 30 seconds. The feature (maximal number of collected coins) is the number of collected coins in the test.

FIG. 14 shows the correlation of the clinical anchor test and the results from the collect the coins test (maximal number of collected coins). The sensor feature results are in clear association with the clinical anchor (pick up tennis ball, then turn hand) in both studies.

### Example 8

Pressure Dataset Acquisition Using a Computer-Implemented Test for Determining Finger Strength (Test: Ring the Bell), a Distal Motor Function Test

TABLE 10

| | | Spearman | Spearman | | | | | |
|---|---|---|---|---|---|---|---|---|
| | | correlation | correlation | P-values | P-value | N | ICC | N ICC |
| feature | | OLEOS | Jewelfish | OLEOS | Jewelfish | OLEOS | OLEOS | OLEOS |
| touch_pressure_mean | Mean touch | 0.635 | 0.907 | 0.003 | 0 | 20 | 0.856 | 16 |
| | pressure | | | | | | | |
| touch_pres_sure_mean | Mean touch | 0.469 | 0.8987 | 0.037 | 0 | 20 | 0.856 | 16 |
| | pressure | | | | | | | |
| percentage | | 0.591 | 0.86078 | 0.006 | 0 | 20 | 0.7036 | 16 |
| touch_pressure_mean | Mean touch | 0.481 | 0.8537 | 0.032 | 0 | 20 | 0.856 | 16 |
| | pressure | | | | | | | |
| percentage | | 0.489 | 0.795 | 0.029 | 0 | 20 | 0.704 | 16 |
| touch_pressure_cv | Coefficient | −0.544 | −0.791 | 0.013 | 0 | 20 | 0.9534 | 16 |
| | of variation | | | | | | | |
| | of touch | | | | | | | |
| | pressure | | | | | | | |
| percentage | | 0.5036 | 0.786 | 0.024 | 0 | 20 | 0.704 | 16 |
| touch_pressure_std | Standard | −0.545 | −0.747 | 0.013 | 0 | 20 | 0.950 | 16 |
| | deviation of | | | | | | | |
| | touch | | | | | | | |
| | pressure | | | | | | | |
| touch_pressure_std | Standard | −0.515 | −0.633 | 0.02 | 0.004 | 20 | 0.950 | 16 |
| | deviation of | | | | | | | |
| | touch | | | | | | | |
| | pressure | | | | | | | |
| touch_pressure_cv | Coefficient | −0.503 | −0.615 | 0.024 | 0.005 | 20 | 0.953 | 16 |
| | of variation | | | | | | | |
| | of touch | | | | | | | |
| | pressure | | | | | | | |
| touch_N | Number of | −0.462 | −0.5975 | 0.04 | 0.007 | 20 | 0.855 | 16 |
| | touches | | | | | | | |
| touch_pressure_mean | Mean touch | 0.528 | 0.470 | 0.017 | 0.042 | 20 | 0.856 | 16 |
| | pressure | | | | | | | |

Covariate:
1: TOTAL 32;
2: MFM17;
3: MFM20;
4: AGEIC
ICC: Intraclass Correlation Coefficient

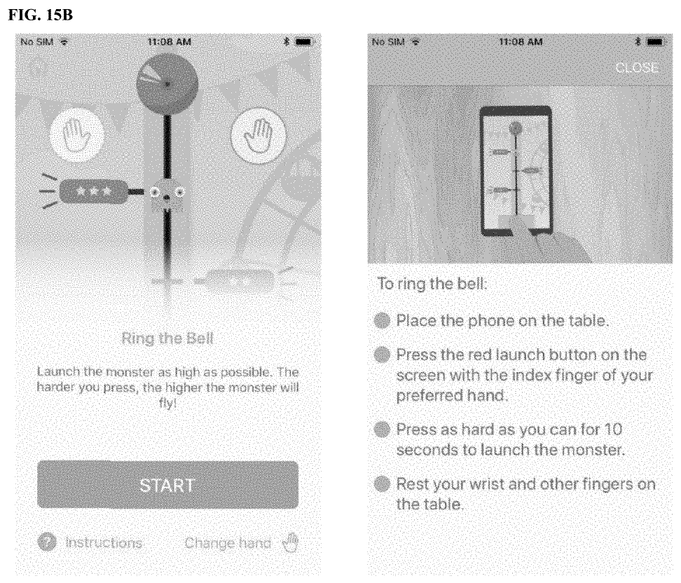

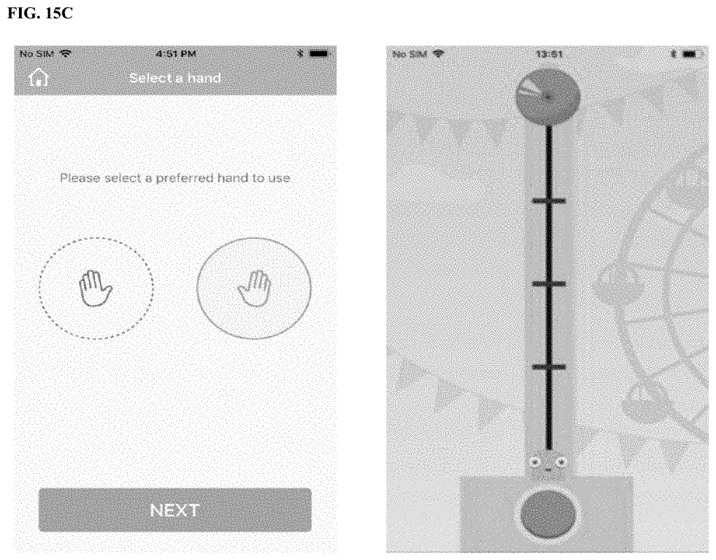

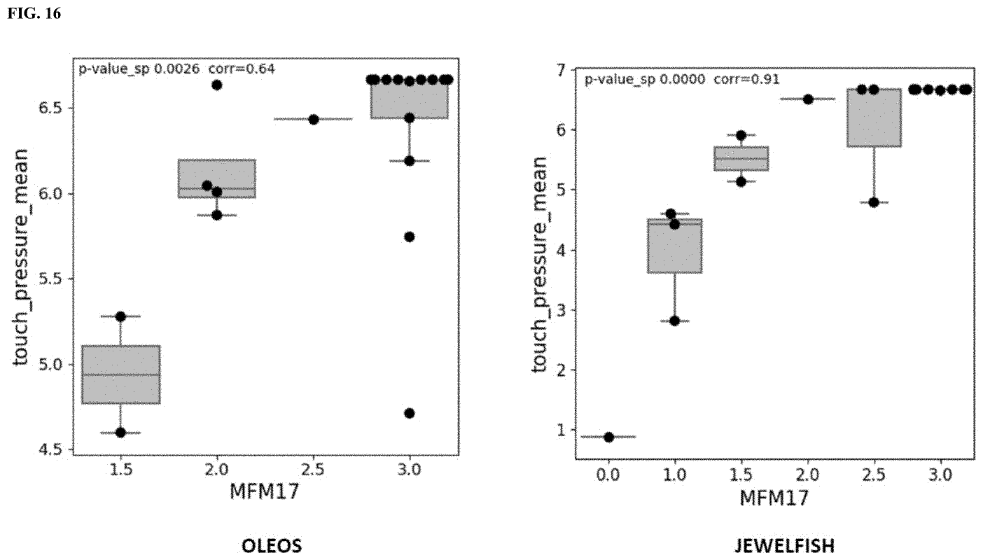

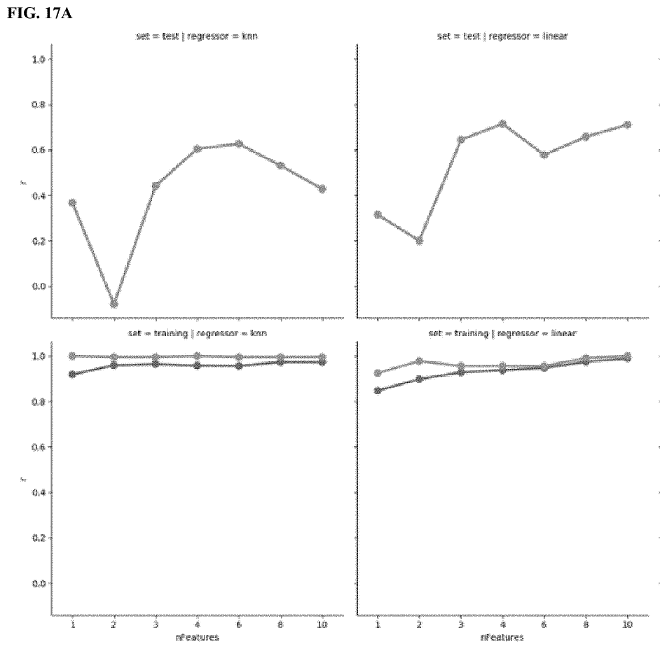

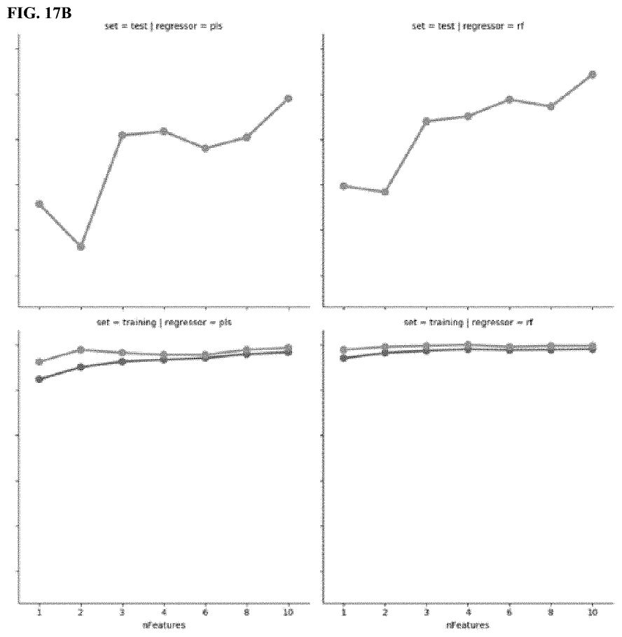

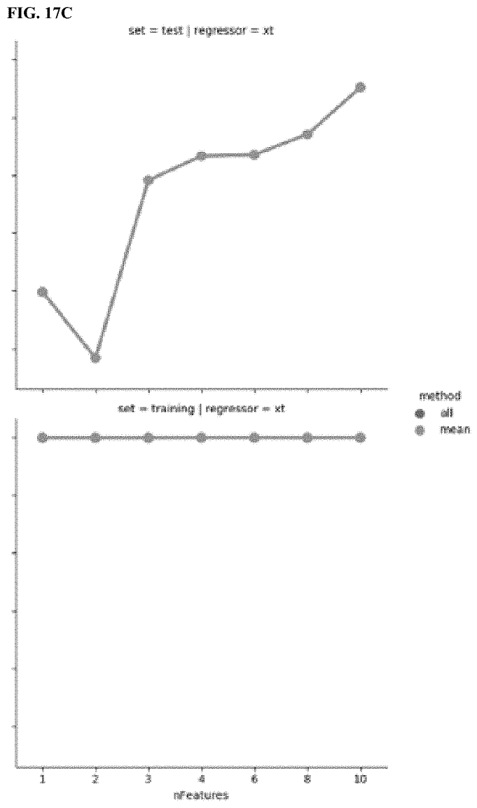

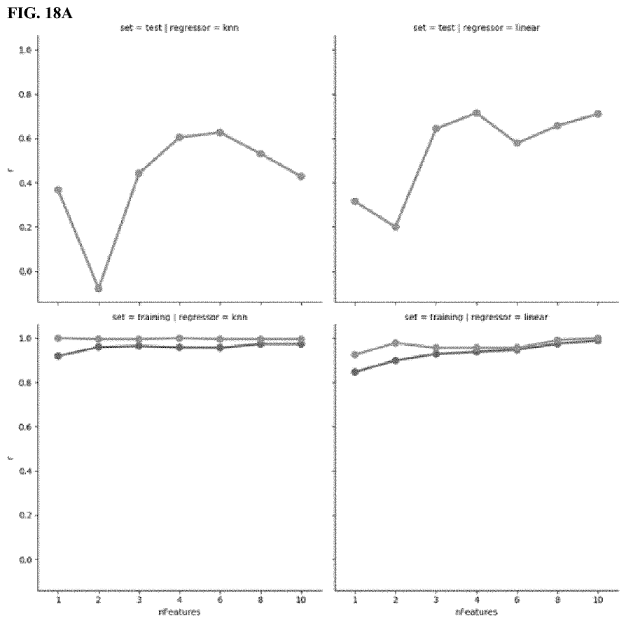

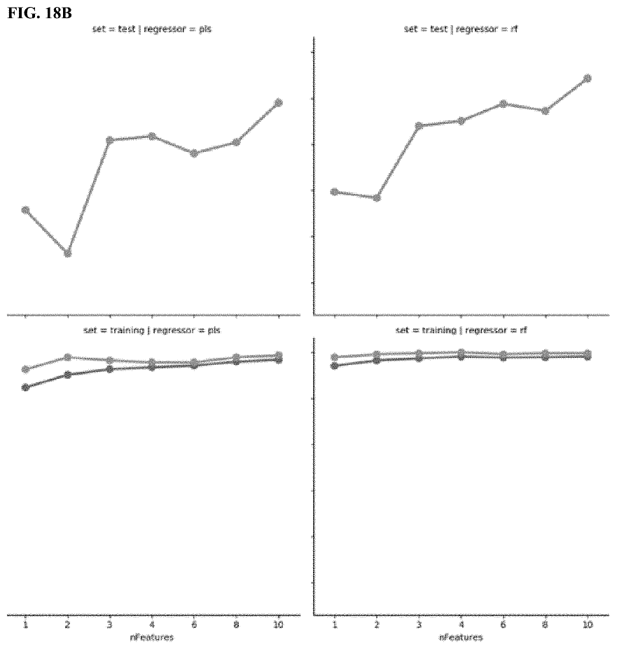

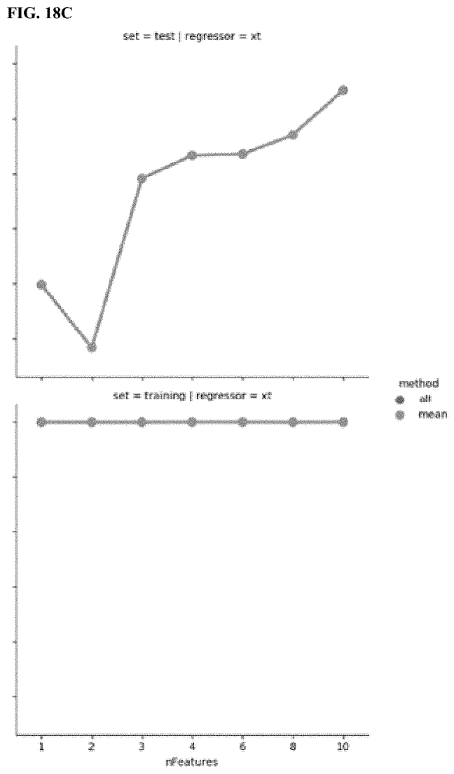

A test for measuring pressure exert by a finger was implemented on a mobile phone (iPhone); see FIG. 15-16. The phone should be placed on the table. The patients shall exert maximum pressure on the surface of the display such that the bell will ring. This means the launch button on the screen should be pressed with the index finger of the preferred hand as hard as possible for at least 10 seconds. Wrist and other fingers should be rest on the table. The test was adapted to measure pressure application by a finger of a patient. The patient needs to play a game aiming to obtain maximum pressure and the duration of maximum pressure application. The test required calibration with respect to the maximum pressure which can be applied by a finger of the subject first. The results of the ring-a-bell test are expressed as percentage of said maximum pressure. The test lasts 10 seconds.

FIG. 16 shows the correlation of the clinical anchor test and the results from the ring the bell test (mean touch pressure exerted during game). The sensor feature results are in clear association with the clinical anchor (pick up 10 coins with one hand in 20 seconds) in both studies.

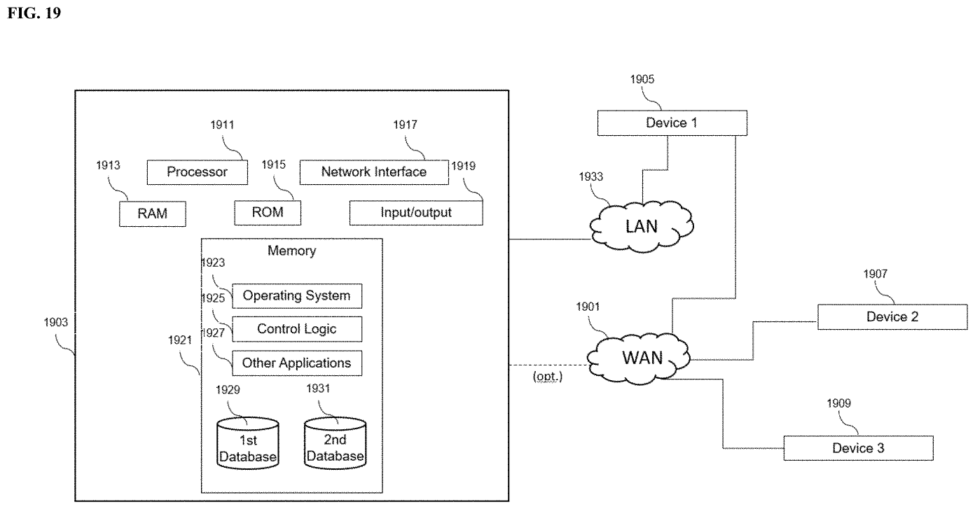

FIG. 19 illustrates one example of a network architecture and data processing device that may be used to implement one or more illustrative aspects described herein. Various network nodes 303, 305, 307, and 309 may be interconnected via a wide area network (WAN) 301, such as the Internet. Other networks may also or alternatively be used, including private intranets, corporate networks, LANs, wireless networks, personal networks (PAN), and the like. Network 301 is for illustration purposes and may be replaced with fewer or additional computer networks. A local area network (LAN) may have one or more of any known LAN topology and may use one or more of a variety of different protocols, such as Ethernet. Devices 303, 305, 307, 309 and other devices (not shown) may be connected to one or more of the networks via twisted pair wires, coaxial cable, fiber optics, radio waves or other communication media.

The term “network” as used herein and depicted in the drawings refers not only to systems in which remote storage devices are coupled together via one or more communication paths, but also to stand-alone devices that may be coupled, from time to time, to such systems that have storage capability. Consequently, the term “network” includes not only a “physical network” but also a “content network,” which is comprised of the data—attributable to a single entity—which resides across all physical networks.

The components may include data server 303, web server 305, and client computers 307, 309. Data server 303 provides overall access, control and administration of databases and control software for performing one or more illustrative aspects described herein. Data server 303 may be connected to web server 305 through which users interact with and obtain data as requested. Alternatively, data server 303 may act as a web server itself and be directly connected to the Internet. Data server 303 may be connected to web server 305 through the network 301 (e.g., the Internet), via direct or indirect connection, or via some other network. Users may interact with the data server 303 using remote computers 307, 309, e.g., using a web browser to connect to the data server 303 via one or more externally exposed web sites hosted by web server 305. Client computers 307, 309 may be used in concert with data server 303 to access data stored therein, or may be used for other purposes. For example, from client device 307 a user may access web server 305 using an Internet browser, as is known in the art, or by executing a software application that communicates with web server 305 and/or data server 303 over a computer network (such as the Internet). In some embodiments, the client computer 307 may be a smartphone, smartwatch or other mobile computing device, and may implement a diagnostic device. In some embodiments, the data server 303 may implement a server.

Servers and applications may be combined on the same physical machines, and retain separate virtual or logical addresses, or may reside on separate physical machines. For example, services provided by web server 305 and data server 303 may be combined on a single server.

Each component 303, 305, 307, 309 may be any type of known computer, server, or data processing device. Data server 303, e.g., may include a processor 311 controlling overall operation of the rate server 303. Data server 303 may further include RAM 313, ROM 315, network interface 317, input/output interfaces 319 (e.g., keyboard, mouse, display, printer, etc.), and memory 321. I/O 319 may include a variety of interface units and drives for reading, writing, displaying, and/or printing data or files. Memory 321 may further store operating system software 323 for controlling overall operation of the data processing device 303, control logic 325 for instructing data server 303 to perform aspects described herein, and other application software 327 providing secondary, support, and/or other functionality which may or may not be used in conjunction with other aspects described herein. The control logic may also be referred to herein as the data server software 325. Functionality of the data server software may refer to operations or decisions made automatically based on rules coded into the control logic, made manually by a user providing input into the system, and/or a combination of automatic processing based on user input (e.g., queries, data updates, etc.).

Memory 321 may also store data used in performance of one or more aspects described herein, including a first database 329 and a second database 331. In some embodiments, the first database may include the second database (e.g., as a separate table, report, etc.). That is, the information can be stored in a single database, or separated into different logical, virtual, or physical databases, depending on system design. Devices 305, 307, 309 may have similar or different architecture as described with respect to device 303. Those of skill in the art will appreciate that the functionality of data processing device 303 (or device 305, 307, 309) as described herein may be spread across multiple data processing devices, for example, to distribute processing load across multiple computers, to segregate transactions based on geographic location, user access level, quality of service (QoS), etc.

One or more aspects described herein may be embodied in computer-usable or readable data and/or computer-executable instructions, such as in one or more program modules, executed by one or more computers or other devices as described herein. Generally, program modules include routines, programs, objects, components, data structures, etc. that perform particular tasks or implement particular abstract data types when executed by a processor in a computer or other device. The modules may be written in a source code programming language that is subsequently compiled for execution, or may be written in a scripting language such as (but not limited to) HTML or XML. The computer executable instructions may be stored on a computer readable medium such as a hard disk, optical disk, removable storage media, solid state memory, RAM, etc. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like. Particular data structures may be used to more effectively implement one or more aspects, and such data structures are contemplated within the scope of computer executable instructions and computer-usable data described herein.

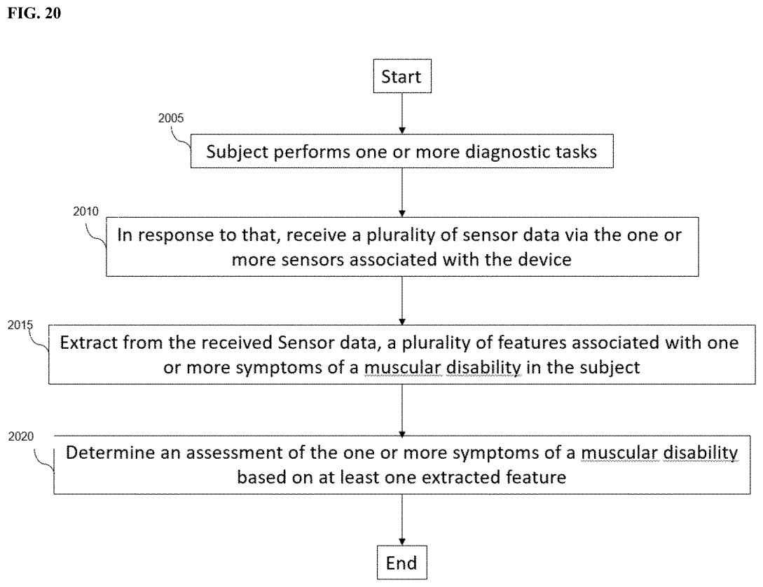

FIG. 20 sets forth an example method for assessing the motor function of a muscular disability, in particular SMA based on active testing of the subject. The method begins by proceeding to step 205, which includes prompting the subject to perform the diagnostic task. In some embodiments, the diagnostic tasks are anchored in or modelled after well-established methods and standardized tests for evaluating and assessing a muscular disability, in particular SMA.

The method proceeds to step 210, which includes in response to the subject performing the one or more diagnostics tasks, receiving, a plurality of second sensor data via the one or more sensors. In response to the subject performing the one or more diagnostic tasks, the diagnostic device receives, a plurality of sensor data via the one or more sensors associated with the device. The method proceeds to step 215, including extracting, from the received sensor data, a second plurality of features associated with the axial motor function of a muscular disability, in particular SMA.

The method proceeds to step 220, which includes determining an assessment of the axial motor function of a muscular disability, in particular SMA based on at least the extracted sensor data.

As discussed above, assessments of symptom severity and progression of a muscular disability, in particular SMA using diagnostics according to the present disclosure correlate sufficiently with the assessments based on clinical results and may thus replace clinical subject monitoring and testing. Diagnostics according to the present disclosure were studied in a group of subject with a muscular disability, in particular SMA subjects. The subjects were provided with a smartphone application that included one or more motor function tests.

1. A method of assessing spinal muscular atrophy (SMA) in a subject comprising the steps of:
a) determining at least one parameter from a dataset of sensor measurements from said subject using a mobile device; and
b) comparing the determined at least one parameter to a reference, whereby SMA is assessed from the result of the comparison.

2. The method of claim 1, wherein the said at least one parameter is a parameter indicative for distal motor function, central motor function, or axial motor function.

3. The method of claim 1, wherein the dataset of sensor measurements of the individual motor function comprises data from the measurement the maximal pressure which can be exerted by a subject with an individual finger or for the capability of exerting pressure with an individual finger over time, the measurement the maximal duration of the tone “aaah”, the maximal amount of touching the screen in a defined time period, in particular within 30 sec, the maximal double touch asynchronity, the variability of acceleration after wind, the number of a thing collected, in particular collected coins and/or the maximal turn speed of the hand.

4. The method of claim 1, wherein the dataset of sensor measurements of the individual motor function comprises data from the following feature measurements:
i. mean pressure applied,
ii. pitch variability,
iii. median time to hit the screen,
iv. double touch asynchronity,
v. time to draw a shape,
vi. maximum turning speed of the phone,
vii. variability of acceleration (after wind), and/or
viii. number of collected coins.

5. The method of claim 1, wherein the dataset of sensor measurements of the individual motor function comprises data from the following feature test:
i. Ring the bell,
ii. Cheer the monster,
iii. Tap the monster,
iv. Squeeze the tomato,
v. Walk the trails,
vi. Turn the phone,
vii. Walk the rope, and/or
viii. Collect the coins.

6. The method of claim 1, wherein the dataset of sensor measurements of the individual motor function comprises data from daily or at least from measurements of every other day, in particular wherein the dataset of sensor measurements of the individual motor function comprises data from sensor measurements obtained in the morning.

7. The method of claim 1, wherein said mobile device has been adapted for carrying out on the subject one or more of the sensor measurements referred to in claim 3.

8. The method of claim 1, wherein a determined at least one parameter being essentially identical compared to the reference is indicative for a subject with SMA.

9. A mobile device comprising a processor, at least one pressure sensor and a database as well as software which is tangibly embedded to said device and, when running on said device, carries out the method of claim 1.

10. A system comprising a mobile device comprising at least one pressure sensor and a remote device comprising a processor and a database as well as software which is tangibly embedded to said device and, when running on said device, carries out the method of claim 1, wherein said mobile device and said remote device are operatively linked to each other.

11. Use of the mobile device according to claim 9 for assessing SMA on a dataset of sensor measurements of the individual subject.

12. A combination of the method according to claim 1 with a pharmaceutical agent suitable to treat SMA in a subject, in particular a m7GpppX Diphosphatase (DCPS) Inhibitors, Survival Motor Neuron Protein 1 Modulators, SMN2 Expression Inhibitors, SMN2 Splicing Modulators, SMN2 Expression Enhancers, Survival Motor Neuron Protein 2 Modulators or SMN-AS1 (Long Non-Coding RNA derived from SMN1) Inhibitors, more particular Nusinersen, Onasemnogene abeparvovec, Risdiplam or Branaplam.

13. A pharmaceutical agent suitable to treat SMA in a subject, in particular a m7GpppX Diphosphatase (DCPS) Inhibitors, Survival Motor Neuron Protein 1 Modulators, SMN2 Expression Inhibitors, SMN2 Splicing Modulators, SMN2 Expression Enhancers, Survival Motor Neuron Protein 2 Modulators or SMN-AS1 (Long Non-Coding RNA derived from SMN1) Inhibitors, more particular Nusinersen, Onasemnogene abeparvovec, Risdiplam or Branaplam wherein the disease of the subject being treated is monitored with a method according to claim 1.

14. A method for the treatment of SMA, wherein the method comprise administering a m7GpppX Diphosphatase (DCPS) Inhibitors, Survival Motor Neuron Protein 1 Modulators, SMN2 Expression Inhibitors, SMN2 Splicing Modulators, SMN2 Expression Enhancers, Survival Motor Neuron Protein 2 Modulators or SMN-AS1 (Long Non-Coding RNA derived from SMN1) Inhibitors, more particular Nusinersen, Onasemnogene abeparvovec Risdiplam or Branaplarn to a subject and wherein the method further comprises a method according to claim 1 to monitor the disease of the subject.

15. A combination of the method according to claim 12, whereby a determined at least one parameter being better compared to the reference parameter of said patient before said subject received treatment with the pharmaceutical agent.

16. A computer-implemented method using machine learning to predict the MFM32 score of a subject suffering from SMA.

17. A computer-implemented method using machine learning to predict the FVC score of a subject suffering from SMA.
